(19) United States
(12) Patent Application Publication
(10) Pub. No.: US 2003/0148982 A1
(43) Pub. Date: Aug. 7, 2003

(54) Bi-spcific chimeric T cells

(76) Inventors: Malcolm K. Brenner, Bellaire, TX (US); Claudia Rossig, Muenster (DE); Cliona Rooney, Bellaire, TX (US)

Correspondence Address: FULBRIGHT & JAWORSKI, LLP, 1301 MCKINNEY, SUITE 5100, HOUSTON, TX, 77010-3095, US

(21) Appl. No.: 10/290,720

(22) Filed: Nov. 8, 2002

Related U.S. Application Data
(60) Provisional application No. 60/337,697, filed on Nov. 13, 2001.

(51) Int. Cl. A61K 48/00; A61K 39/12; C12N 5/08; C12N 15/867

(52) U.S. Cl. 514/44; 424/93.21; 435/372; 435/456

(57) ABSTRACT

The present invention is directed to a bi-specific chimeric T lymphocyte, wherein the lymphocyte comprises both an antigen-specific receptor, such as for Epstein-Barr Virus, and a chimeric receptor, such as for a tumor. In a particular embodiment, administration of an Epstein-Barr Virus T lymphocyte with an 14.G2a-ζ antitumor chimeric receptor is utilized for therapy of neuroblastoma.

This application claims priority to U.S. Provisional Application Serial No. 60/337,697, filed Nov. 13, 2001, and 60/337,697, filed May 30, 2002, both of which are incorporated by reference herein in their entirety.

The work herein was supported by grant NIH CA75014 from the United States Government. The United States Government may have certain rights in the invention.

### FIELD OF THE INVENTION

The present invention is directed to the fields of immunology, cancer and cell biology. Specifically, the present invention is directed to methods and compositions for an antigen-specific T lymphocyte having a chimeric receptor. More specifically, the present invention is directed to methods and compositions for an Epstein-Barr Virus (EBV)-specific T lymphocyte having a chimeric receptor.

### BACKGROUND OF THE INVENTION

The genetic modification of human T cells to express tumor antigen-specific chimeric receptors is an attractive means of providing large numbers of effector cells for adoptive immunotherapy. The primary mechanisms by which tumor cells escape from immune recognition, such as downregulation of major histocompatibility complex (MHC) molecules, are efficiently bypassed through use of this strategy. T lymphocytes engineered to express the recombinant receptor genes are capable of both specific lysis and cytokine secretion upon exposure to tumor cells expressing the requisite target antigen (Eshhar et al., 1993; Stancovski et al., 1993).

Although adoptive transfer of chimeric receptor-expressing peripheral blood-derived T lymphocytes has produced some antitumor activity in mice (Altenschmidt et al., 1997; Hwu et al., 1995; McGuinness et al., 1999), clinical results have been disappointing (Brocker and Karjalainen, 1995; Brocker, 2000). The most pertinent issue is that chimeric T cells fail to expand and rapidly lose their function in vivo. Activation studies performed in transgenic mice suggest that the function of chimeric receptor proteins depends upon the activation status of the T cell (Krause et al., 1998; Khanna and Burrows, 2000). Signaling through chimeric T cell receptors alone was shown to be insufficient to induce proliferation and effector function in primary T lymphocytes, unless they had been prestimulated through their native receptor (Krause et al., 1998; Khanna and Burrows, 2000). Even under these conditions, responsiveness was soon lost. This problem is accentuated by the general lack of tumor cell costimulatory molecules essential for the induction and maintenance of a T cell response (Heslop et al., 1996). The development of strategies to prevent functional inactivation of chimeric receptor-modified cells in vivo would greatly enhance their therapeutic value.

Thus, expression and functional activation of a chimeric receptor specific for tumor cells or pathogen-infected cells, such as HIV-infected cells, has been demonstrated. Specifically, Eshhar et al. (1993) describes specific activation and targeting of mouse hybridoma MD.45 or MD.27J CTLs through single chains of chimeric receptor consisting of antibody-binding domains and the γ or ζ subunits of T-cell receptors.

WO 00/31239 describes immune cells having a predefined specificity, wherein the cell is complexed either with an antigen-specific MHC-restricted chimeric T cell receptor or is transfected with an antigen-specific MHC-restricted chimeric TCR gene. In specific embodiments, the chimeric T cell receptor comprises a scFv T cell receptor. In other specific embodiments, the immune cell is a T lymphocyte.

WO 93/19163 is directed to chimeric genes encoding a scFv domain of a specific antibody, a transmembrane domain, and a cytoplasmic domain of, in some embodiments, a T cell receptor. Also described are methods of treating tumors using lymphocyte cells transformed with vectors comprising the chimeric genes.

U.S. Pat. No. 5,359,046 describes chimeric DNA and cells transfected therewith wherein the DNA encodes a membrane bound protein comprising a signal sequence, a non-MHC restricted extracellular binding domain of a surface membrane protein, such as a scFv that binds to a ligand on a cell surface or viral protein, a transmembrane domain, particularly from CD4, CD8, and so on, and a cytoplasmic signal-transducing domain of a protein that activates an intracellular messenger system, such as CD3ζ chain, and the like.

Immune regulation of latent EBV infection is one of the best-studied examples of persistent T cell-mediated immune control. More than 90% of adults are seropositive for this virus, and their B cells expressing EBV-encoded latency-associated transforming proteins are tightly controlled by high levels of EBV-specific HLA-restricted cytotoxic T cells (CTLs), which persist indefinitely (Roskrow et al., 1998). The interaction of specific T lymphocytes with the target cells of latent EBV infection in immunocompetent hosts is characterized by a complex self-modulating network of cellular immune-mediated interactions, resulting in potent target cell lysis. These EBV-specific immune responses can be reconstituted by transfusion of in vitro-generated EBV-specific CTL lines into patients with EBV-associated infections and malignancies (Rooney et al., 1995; Rooney et al., 1998). The transfused T lymphocytes show a high initial degree of in vivo expansion, and contain all necessary subpopulations to produce regression of even bulky EBV+ tumors. Gene marking studies have demonstrated their persistence for more than 6 years with retained ability to respond to viral stimulation in vivo (Rooney et al., 1995; Rooney et al., 1998; Schulz et al., 1984; Mujoo et al., 1987).

The rapid expansion of EBV-specific T cells in vivo and their persistence in a functional state, life-long without further immunization, make them attractive candidates for tumor cell targeting via chimeric T cell receptors. There is a significant absence in the art for the demonstration of chimeric receptor T cells to persist long term in vivo (such as if CD4 and CD8 T cells are present), to destroy the pathogen-infected cell, and to permit the T cells to expand in vivo to large numbers without toxicity. In particular, these characteristics are met with the novel chimeric receptor-bearing specific T lymphocytes, such as the Epstein-Barr Virus-specific T lymphocytes, and methods utilized therewith.

### SUMMARY OF THE INVENTION

The present invention is directed to the following embodiments:

An embodiment of the invention is a T lymphocyte, comprising an antigen-specific receptor as well as a chimeric receptor, wherein the presence of said antigen-specific receptor leads to increased in vivo survival of said lymphocyte. In one embodiment, the antigen specific receptor recognizes a viral polypeptide. In another embodiment, the chimeric receptor comprises an antigen-binding moiety, such as a single chain antibody. In a further embodiment, the T lymphocyte expresses a native T cell receptor specific for Epstein Barr Virus, and a chimeric receptor specific to an anitumor antigen. In a specific embodiment, the antitumor chimeric receptor is 14.G2a-ζ. In another specific embodiment, the antitumor chimeric receptor is specific for CD19.

Another embodiment of the present invention is a population of cytotoxic T lymphocytes, comprising at least one cytotoxic T lymphocyte having an antigen-specific receptor as well as a chimeric receptor, wherein the presence of said antigen-specific receptor leads to increased in vivo survival of said lymphocyte. In a specific embodiment, the population of lymphocytes comprises CD4⁺ T lymphocytes, CD8⁺ T lymphocytes, or a combination thereof.

An embodiment of the present invention is a method of enhancing activity of a chimeric T lymphocyte in an individual. In this embodiment, a T lymphocyte is obtained, wherein said T lymphocyte comprises an antigen-specific receptor. The presence of said antigen-specific receptor leads to increased in vivo survival of said lymphocyte; the lymphocyte also comprising a chimeric receptor. Part of this embodiment includes administering said T lymphocyte to an individual. In a specific embodiment, the antigen which the antigen-specific receptor is directed to is an Epstein Barr Virus polypeptide.

Another embodiment of the invention is a method of treating a disease in an individual, wherein said disease is associated with a pathogen or cell having a first antigen. This embodiment comprises obtaining a cytotoxic T lymphocyte, wherein said lymphocyte comprises a receptor specific for a second antigen. The presence of the second antigen leads to increased in vivo survival of said lymphocyte. The cytotoxic T lymphocyte of this embodiment also comprises a chimeric receptor specific for said first antigen. Another aspect of this embodiment comprises administering said T lymphocyte to said individual. In a specific embodiment of this invention, said disease is cancer and said first antigen is a tumor-specific or tumor-associated antigen, and said second antigen is to an Epstein Barr Virus polypeptide.

In one embodiment of the present invention, a method of treating a tumor in an individual is described. The method comprises obtaining a cytotoxic T lymphocyte, wherein said lymphocyte comprises an antigen-specific receptor, wherein the presence of said antigen-specific receptor leads to increased in vivo survival of said lymphocyte. In such an embodiment, the cytotoxic T lymphocyte also comprises an antitumor chimeric receptor. Further, the method comprises administering said cytotoxic T lymphocyte to an individual. In a specific embodiment of the invention, the antigen specific receptor, which increases in vivo survival of said lymphocyte, is specific for Epstein Barr Virus. In a further specific embodiment, said T lymphocyte may be obtained by means of transfecting a vector comprising a polynucleotide encoding said chimeric receptor into a T lymphocyte. In a yet further specific embodiment, said vector is a retroviral vector. In one embodiment, the antitumor chimeric receptor is 14.G2a-ζ and the treated tumor is of neural crest origin and is neuroblastoma or ganglioneuroma. In another embodiment, the tumor is from lung cancer, melanoma, breast cancer, prostate cancer, colon cancer, or lymphoma. In a further specific embodiment, the lyphoma is of B cell origin and the antitumor chimeric receptor is CD19 specific. Yet another specific embodiment comprises administering to said individual an additional cancer therapy. The additional cancer therapy may be chemotherapy, radiation, surgery, or a combination thereof.

An embodiment of the invention is a method of preventing cancer or an intractable infection in an individual. In this embodiment, said cancer or intractable infection is associated with a pathogen or cell having a first antigen, and it comprising administering to an individual susceptible to said cancer or intractable infection at least one cytotoxic T lymphocyte. Said lymphocyte comprises a receptor specific for a second antigen, wherein the presence of said second antigen-specific receptor leads to increased in vivo survival of said lymphocyte; and a chimeric receptor specific for said first antigen. The first antigen, in a specific embodiment, is an Epstein Barr Virus polypeptide, and the cancer may be of neural crest origin, lung cancer, melanoma, breast cancer, prostate cancer, colon cancer, or lymphoma. The intracable infection, in a specific embodiment, is a viral infection or a bacterial infection. In a further specific embodiment, the viral infection is acquired immunodeficiency syndrome (AIDS), hepatitis B or hepatitis C.

One embodiment of the invention is a kit, housed in a suitable container, comprising at least one cytotoxic T lymphocyte in a pharmaceutically acceptable solution. The cytotoxic T lymhpocyte in this embodiment, as in the above embodiments, comprises a receptor specific for a second antigen, wherein the presence of said second antigen-specific receptor leads to increased in vivo survival of said lymphocyte, and a chimeric receptor specific for said first antigen. In a specific embodiment, the second antigen is an Epstein Barr Virus polypeptide.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates the structure of the T cell receptor complex (top), and chimeric receptor expressed as part of that complex

FIGS. 2A through 2D show flow cytometric analysis indicating transduced CTLs express surface 14.G2a-ζ chimeric receptors. FIGS. 2A and 2C represent nontransduced CTLs, and FIGS. 2B and 2D represent transduced CTLs.

FIG. 3A demonstrates that 14.G2a-ζ transduced CTLs proliferate in response to EBV but not to tumor targets. 14.G2a-ζ transduced (CTL/chRec) and nontransduced EBV-specific CTLs (CTL/NT) were stimulated with irradiated (40 Gy) autologous or mismatched allogeneic LCLs, or with G_{D2}⁺ (LAN-1) or G_{D2}⁻ (A-204) tumor cells at a 1:4 stimulator to responder ratio. Proliferative responses were assessed by measurement of [³H] thymidine uptake.

FIG. 3B shows that 14.G2a-ζ transduced CTLs expand in response to EBV-LCLs but not tumor targets. EBV-specific CTL cultures, either nontransduced or transduced with the chimeric receptor gene 14.G2a-ζ or with a control gene encoding enhanced green fluorescent protein (EGFP), received weekly stimulations with irradiated (40 Gy) autologous LCL or G_{D2}⁺ tumor cell targets at a 1:4 stimulator-to-responder ratio. Cells were fed twice weekly with medium containing rhuIL-2 (40 IU/ml), and their growth was assessed. A representative experiment (of four) is shown.

FIG. 4 shows IFN-γ and GM-CSF release by CTLs in response to coincubation with tumor target cells. Non-transduced (NT) CTLs or CTLs transduced with SFG/14.G2a-ζ were cocultured with tumor cells at a 3:1 target-to-effector ratio for 24 hours. LAN-1 is a G_{D2}⁺ neuroblastoma cell line and A-204 is a G_{D2}⁻ rhabdomyosarcoma cell line. Numbers above columns indicate the amount of cytokine secretion in response to autologous LCL targets for each population. Data shown are representative of independent experiments performed with CTL lines from three donors.

FIG. 5 demonstrates lack of MHC restriction in chRec-mediated tumor cell killing by transduced CTL. ⁵¹Cr-labeled G_{D2}⁺ LAN-5 neuroblastoma cells and autologous EBV-LCL were preincubated for 30 minutes with monoclonal antibodies recognizing monomorphic determinants of HLA class I or HLA class II, then coincubated for 4 hours with 14.G2a-ζ-transduced CTL at a 20:1 effector-to-target ratio.

FIGS. 6A through 6C demonstrate cytolytic activity of three EBV-specific CTL lines against EBV and tumor targets. Transduced CTLs were tested against ⁵¹Cr-labeled autologous LCLs, class I mismatched allogeneic LCLs, G_{D2}⁺ neuroblastoma tumor cells (LAN-1), G_{D2}⁻ rhabdomyosarcoma cells (A-204), or autologous phytohemagglutinin-stimulated lymphoblasts. FIG. 6A shows 14.G2a-ζ and 14.G2a-ζ-transduced CTL line #2; FIG. 6B shows 14.G2a-ζ-transduced CTL line #4; and FIG. 6C shows 14.G2a-ζ-transduced CTL line #8.

FIGS. 7A and 7B illustrate a cold target inhibition assay, wherein 14.G2a-ζ transduced EBV-specific CTLs were preincubated with unlabeled autologous LCL (Auto-LCL), HLA-mismatched allogeneic LCL (Allo LCL), G_{D2}⁺ (LAN-1) or G_{D2}⁻ (A-204) tumor cells at various cold to hot target ratios. Cytotoxic activity was then determined against ⁵¹Cr-labeled autologous LCL (FIG. 7A) and G_{D2}⁺ LAN-1 tumor cells (FIG. 7B) at an effector to target ratio of 40:1.

FIGS. 8A and 8B show LMP-2/HLA-A2 tetramer+ CTLs coexpress the 14.G2a-ζ chimeric receptor. On day 14 post-transduction, CTLs were stained with monoclonal antibody 1A7 and FITC-labeled goat antimouse antibody, then incubated with PE-labeled LMP-2/HLA-A2 tetramer, followed by staining with PerCP-labeled anti-CD8 antibody. One million events were acquired and analyzed. Indicated are the absolute numbers of events for tetramer-positive cells that express detectable levels of 14.G2a-ζ in a population of nontransduced CTLs (FIG. 8A) or 14.G2a-ζ transduced CTLs (FIG. 8B).

FIGS. 9A and 9B show transduced cells are induced to proliferate by stimulation with autologous EBV-LCL. 14.G2a-ζ transduced (FIG. 9A) and nontransduced (FIG. 9B) EBV-specific CTLs were stimulated with irradiated autologous (Auto) or mismatched allogeneic (Allo) LCLs, or with G_{D2}⁺ (LAN-1) or G_{D2}⁻ (A-204) tumor cells at a 1:4 stimulator to responder ratio. After 7-14 days, CTLs were stimulated with autologous LCLs (Auto/Auto, LAN-1/Auto, JF/Auto, A-204/Auto, Allo/Auto) or stimulated as before (LAN-1/LAN-1, JF/JF, A-204/A-204, Allo/Allo). Proliferative responses were then assessed by measurement of [³H] thymidine uptake. Shown is one representative experiment of two.

FIG. 10 illustrates an exemplary model of use of EBV-infected B lymphocytes and chimeric T cell receptors (TCRs) to target cancer cells. In this model, CD8 T cells bearing a G_{D2}-specific chimeric TCR are activated by EBV antigen binding to a native TCR and are costimulated through the interaction of B7/CD28. They may receive additional cognate help from EBV-specific CD4⁺ T cells. The stimulated chimeric receptor positive cells are able to recognize and lyse G_{D2}-positive tumor cells (such as neuroblastoma) via the corresponding epitope of the chimeric TCR.

FIG. 11 shows that CTL phenotype is unchanged after transduction. Nontransduced and CD19ζ-transduced, CTL were stained with fluorescence-labeled antibodies against T cell surface antigens CD3, CD4, CD8, and CD56, and surface immunofluorescence was analyzed by flow cytometry.

FIG. 12 demonstrates that CD19ζ-transduced EBV-specific CTL specifically lyse both EBV targets and CD19⁺ tumor cells. Seven EBV-specific CTL lines generated from 4 individual donors were transduced with CD19ζ, and both nontransduced and transduced CTL were tested against ⁵¹Cr-labeled autologous LCL, class I mismatched allogeneic LCL, CD19⁺ tumor cells (Raji, Reh), primary leukemic blasts, and against CD19⁻ tumor cells (K-562) in a 4 hr ⁵¹Cr release assay.

FIGS. 13A through 13D show that antibody blocking of target cell lysis by nontransduced and CD19ζ transduced CTL. ⁵¹Cr-labeled Raji cells (FIGS. 13A and 13C), or autologous LCL (FIGS. 13B and 13D) were preincubated with the indicated concentrations of mAb CD19 or with monoclonal antibodies recognizing monomorphic determinants of HLA class I or HLA class II, then coincubated for 4 hours with nontransduced (FIGS. 13B and 13D) or CD19ζ transduced CTL (FIGS. 13A and 13C) at a 20:1 effector-to-target ratio. Shown is one representative experiment of two.

FIGS. 14A through 14D show that antibody blocking of autologous and allogeneic EBV target cell lysis by CD19ζ-transduced CTL. ⁵¹Cr-labeled HLA-mismatched allogeneic (FIGS. 14A and 14B), or autologous LCL (FIGS. 14C and 14D) were preincubated with the indicated concentrations of monoclonal antibodies recognizing monomorphic determinants of HLA class I or HLA class II (FIGS. 14A and 14C) or with mAb CD19 (FIGS. 14B and 14D), then coincubated for 4 hours with CD19ζ transduced CTL at a 20:1 effector-to-target ratio. Shown is one representative experiment of two.

FIGS. 15A and 15B illustrate the results of a cold target inhibition assay. CD19ζ transduced EBV-specific CTL were preincubated with unlabeled autologous LCL (Auto), HLA-mismatched allogeneic LCL (Allo), CD19⁺ (Raji) or D19⁻ (K-562) tumor cells at various cold to hot target ratios. Cytotoxic activity was then determined against ⁵¹Cr-labeled allogeneic LCL (FIG. 15A) and autologous LCL (FIG. 15B) at an effector to target ratio of 20:1. Shown is one representative experiment of three, performed with CTL lines obtained from two donors.

FIGS. 16A and 16B show the activity of CD19ζ transduced CTL. CD19ζ transduced CTL specifically release IFN-γ in response to autologous and mismatched allogeneic EBV-LCL and CD19⁺ tumor targets (FIG. 16A). ζ⁺ EBV-specific CTL clone #11 were stimulated with irradiated autologous or allogeneic LCL from two mismatched donors, or with CD19⁺ (Reh, Daudi) or D19⁻ (Jurkat, A-204) tumor cells at a 3:1 stimulator to responder ratio. Following 72 hr coincubation, the interferon-γ concentration in the supernatants was quantified by ELISA. A representative experiment of two is shown. A clonal population of CD19ζ⁺ CTL obtained by single cell cloning of CD19ζ-transduced bulk CTL was tested against ⁵¹Cr-labeled autologous LCL, class I mismatched allogeneic LCL, CD19⁺ tumor cells (Raji), and against CD19⁻ tumor cells (K-562) in a 4 hr ⁵¹Cr release assay (FIG. 16B).

FIGS. 17A and 17B show that CD19ζ transduced CTL specifically proliferate in response to autologous and mismatched allogeneic EBV but not CD19⁺ tumor targets. Cells of CD19ζ⁺ (FIG. 17A) and a CD19ζ⁻ (FIG. 17B) EBV-specific CTL clone were stimulated with irradiated autologous LCL, or with allogeneic LCL from three mismatched donors, or with CD19⁺ (Reh, Daudi) or D19⁻ (K-562) tumor cells at a 1:4 stimulator to responder ratio. Proliferative responses were assessed by measurement of [³H] thymidine uptake after 72 hr coincubation. A representative experiment of three is shown.

FIG. 18A shows CD19ζ transduced bulk CTL expand in response to autologous and allogeneic LCL but not to CD19⁺ tumor targets. EBV-specific, nontransduced (FIG. 18A; panels A and C) and CD19ζ transduced CTL (FIG. 18A; panels B and D) were weekly stimulated with irradiated (40 Gy) autologous LCL, HLA-mismatched allogeneic LCL or CD19⁺ tumor cells in the presence or absence of immobilised CD28-specific monoclonal antibody (1 μg/ml) (FIG. 18A; panels C and D), and their growth was assessed. Experiments were reproduced with a total of four CTL lines from 3 donors.

FIG. 18B shows repeated assessment of transgene copy number in CD 19ζ-transduced CTL receiving weekly stimulations with mismatched allogeneic LCL. Quantification was performed by PCR detection of a provirus LTR segment in genomic DNA extracted from the transduced CTL each week prior to restimulation.

FIG. 18C shows cytolytic activity of an EBV-specific CTL line against EBV and tumor targets. CTL transduced with the CD19ζ gene were tested against ⁵¹Cr-labeled autologous LCL, class I mismatched allogeneic LCL, CD19⁺ tumor cells (Reh), and against CD19⁻ tumor cells (K-562) in a 4 hr ⁵¹Cr release assay following eight weekly stimulations with either autologous LCL (FIG. 18C; panel A) or allogeneic LCL (FIG. 18C; panel B).

### DETAILED DESCRIPTION OF THE INVENTION

In keeping with long-standing patent law convention, the words “a” and “an” when used in the present specification in concert with the word comprising, including the claims, denote “one or more.”

The practice of the present invention will employ, unless otherwise indicated, conventional techniques of molecular biology, microbiology, recombinant DNA, and immunology, which are within the skill of the art. Such techniques are explained fully in the literature. See e.g., Sambrook, Fritsch, and Maniatis, MOLECULAR CLONING: A LABORATORY MANUAL, Second Edition (1989), OLIGONUCLEOTIDE SYNTHESIS (M. J. Gait Ed., 1984), ANIMAL CELL CULTURE (R. I. Freshney, Ed., 1987), the series METHODS IN ENZYMOLOGY (Academic Press, Inc.); GENE TRANSFER VECTORS FOR MAMMALIAN CELLS (J. M. Miller and M. P. Calos eds. 1987), HANDBOOK OF EXPERIMENTAL IMMUNOLOGY, (D. M. Weir and C. C. Blackwell, Eds.), CURRENT PROTOCOLS IN MOLECULAR BIOLOGY (F. M. Ausubel, R. Brent, R. E. Kingston, D. D. Moore, J. G. Siedman, J. A. Smith, and K. Struhl, eds., 1987), CURRENT PROTOCOLS IN IMMUNOLOGY (J. E. Coligan, A. M. Kruisbeek, D. H. Margulies, E. M. Shevach and W. Strober, eds., 1991); ANNUAL REVIEW OF IMMUNOLOGY; as well as monographs in journals such as ADVANCES IN IMMUNOLOGY. All patents, patent applications, and publications mentioned herein, both supra and infra, are hereby incorporated herein by reference.

I. Definitions

The term “polypeptide” as used herein refers to any peptide or peptide fragment. This includes polypeptides of viral origin that are translated by the infected cell. In one embodiment of the invention, viral polypeptide fragments are presented on the cell surface and are recognized by T cell receptors.

The term “antigen-binding moiety” as used herein refers to a component of a receptor molecule that provides recognition of at least one receptor-specific antigen.

The term “chimeric receptor” as used herein is defined as a cell-surface receptor comprising the variable domains of the heavy and the light chain (scFv) of an antibody and a constant region of a T-cell receptor.

The term “cytotoxic T lymphocytes” or “CTLs” as used herein refers to T cells which bear the CD3 cell surface determinant and which form the phylogenetic family of lymphocytes that are involved in the cell-mediated lysis of target cells bearing cognate antigens. CTLs include pre-CTLs and effector CTLs. “Pre-CTLs” are virgin or memory T lymphocytes that are committed to proliferating towards or being activated into effector-CTLs upon stimulation by antigen-displaying cells and/or accessory cells. “Effector CTLs” arise from the activation of pre-CTLs, and respond to antigen-bearing target cells by mediating lysis of the target cell. In a preferred embodiment of the present invention, the CTLs are effector CTLs.

Most CTLs are of the CD8+ phenotype, but some CTLs are CD4+. Although most CTLs are generally antigen-specific and MHC-restricted, in that they recognize antigenic peptides only in association with the Major Histocompatibility Complex (MHC) molecules on the surface of target cells, a skilled artisan recognizes that the CTLs of the present invention are independent of this MHC characteristic in recognition of G_{D2}-bearing cells, although stimulation to proliferate is MHC-dependent.

CTLs may be specific for a wide range of viral, tumor or allospecific antigens, including HIV, EBV, CMV and a wide range of tumor antigens. A method for culturing CTLs in vitro is the “Rapid Expansion Method (REM)” described by Stanley Riddell in U.S. patent application Ser. No. 08/299,930, abandoned, filed Aug. 31, 1994, and continuation application Ser. No. 08/317,100, U.S. Pat. No. 5,827,642, filed Oct. 3, 1994 (incorporated herein by reference).

The term “increased in vivo survival” as used herein is defined as an increase in survival of a T lymphocyte over its survival in the absence of an antigen-specific T cell receptor and antigen. The average survival of a chimeric T lymphocyte is 1-12 weeks. An increase in survival would be at least up to several years.

The term “intractable infection” as used herein is defined as an infection by a pathogen which is difficult to alleviate, remedy, or cure. Examples include AIDS, Hepatitis B, Hepatitis C, and chronic EBV infection.

The term “lymphocytes” as used herein refers to cells that specifically recognize and respond to non-self antigens and are responsible for development of specific immunity.

The term “tumor-specific antigen” as used herein refers to an antigen on the surface of malignant cells that may consist of parts that are unique to the cancerous cells and are not present on their normal counterparts.

The term “tumor-associated antigen” as used herein refers to an antigen present on both normal and cancerous cells but ‘hidden’ on normal cells, becoming ‘visible’ when malignant, or overexpressed on the latter, as a product of cellular oncogenes.

The term “tumor of neural crest origin” as used herein is defined as a tumor in cells which have their origin from embryonic cells found in the neural crest. Examples include neuroblastoma, ganglioneuroma, melanoma, and small cell lung carcinoma.

Primary T cells expressing chimeric receptors specific for tumor or viral antigens have considerable therapeutic potential. Unfortunately, their clinical value is limited by their rapid loss of function and failure to expand in vivo, presumably due to the lack of costimulator molecules on tumor cells and the inherent limitations of signaling exclusively through the chimeric receptor. Epstein-Barr virus infection of B lymphocytes is near universal in humans and stimulates high levels of EBV-specific helper and cytotoxic T cells, which persist indefinitely due to the continued presence of viral antigens. It is known that EBV-specific T cells generated in vitro will expand, persist and function for more than 6 years in vivo. The Examples provided herein demonstrate that EBV-specific (but not primary) T cells transduced with tumor-specific chimeric receptor genes can be expanded and maintained long term in the presence of EBV-infected B cells. They recognize EBV-infected targets through their conventional T cell receptor and tumor targets through their chimeric receptors, and they efficiently lyse both. Thus, EBV-specific T cells expressing chimeric antitumor receptors represent a new source of effector cells that would persist and function long term after their transfer to cancer patients.

II. The Present Invention

Adoptive immunotherapy with chimeric receptor-modified T lymphocytes has shown promise in preclinical studies as a means to combat infectious (Roberts et al., 1994; Yang et al., 1997) and malignant diseases (Hwu et al., 1995). However, the first clinical evaluation of chimeric receptor-modified cells revealed a disappointing lack of correlation between in vivo and in vitro cytotoxicity (Walker et al., 2000). One of the major factors limiting successful therapeutic use of modified T cells is their failure to expand and short life-span in vivo, even in the absence of any immune response directed against the chimeric T cells. CD4⁺ helper function plays a crucial role in establishing or maintaining CD8⁺ CTL-mediated antiviral or antitumoral immunity (Cardin et al., 1996; Brodie et al., 1999; Matloubian et al., 1994), and long-term maintenance of engineered T cells is clearly improved if both CD8⁺ and CD4⁺ transduced T cells are infused, rather than CD8 cells alone (Walker et al., 2000; Mitsuyasu et al., 2000). Previous clinical trials in HIV infection have demonstrated prolonged, high-level persistence of chimeric receptor-modified CD4⁺ and CD8⁺ T cells for at least one year. However, no significant mean change in plasma HIV RNA or blood proviral DNA was observed in patients with persisting modified T lymphocytes. An explanation for this observation is that even in the continued presence of detectable chimeric receptor-modified cells in vivo, the surviving T lymphocytes may lose their ability to produce cytokines and to lyse their targets, reflecting functional inactivation of the modified cells. Supporting this concept are studies in a transgenic mouse model which showed that chimeric receptor-mediated signaling was not sufficient to trigger activation of resting primary T cells (Brocker et al., 1995; Brocker, 2000). Although the lack of coreceptor signaling by most tumor targets probably contributes to this effect (Krause et al., 1998), it is also likely that chimeric receptors provide only limited access to downstream signaling pathways (Brocker et al., 1995; Brocker, 2000). The pattern of T cell activation triggered by chimeric receptor engagement observed herein, including efficient target cell lysis, reduced levels of specific cytokine release, and lack of cellular proliferation, is reminiscent of the T cell response to altered peptide ligands as a consequence of incomplete phosphorylation of T cell receptor-associated proximal activation motifs.

Thus, the present invention overcomes deficiencies in the art by employing novel bi-specific chimeric T cells and methods of their use for therapies in disease, such as cancer. It is shown herein that EBV-specific CTLs, as merely an example of a CTL, can be engineered to recognize and lyse tumor cell targets via chimeric receptors while maintaining their ability to proliferate in response to EBV target antigens and to destroy virus-infected cells.

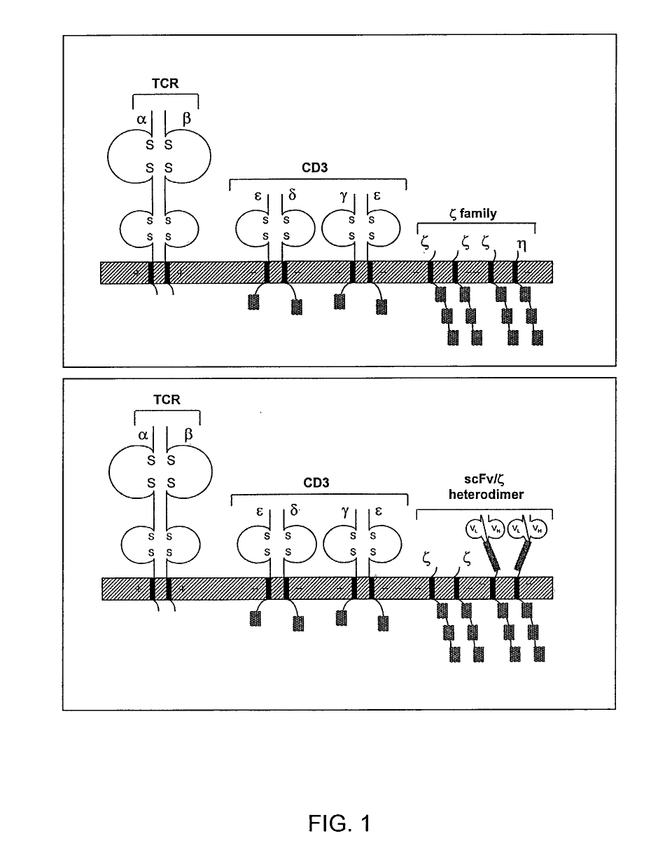

There is growing clinical interest in the use of chimeric T cells for the treatment of cancer and intractable infections (Eshhar et al., 1993; Walker et al., 2000). Such chimeric T cells (see FIG. 1) carry the conventional T cell receptor, but in addition are genetically modified to express a single chain antibody that recognizes cell surface determinants on the malignant/infected cells. The anticipation had been that these chimeric T cells would retain the desirable properties of antibodies (including universal rather than MHC restricted recognition of the target) while processing the cytotoxic and trafficking potential of T lymphocytes. Unfortunately, results from clinical trials have consistently shown that chimeric T cells rapidly lose their activity in vivo. This is because most tumor cells lack the co-stimulator molecules necessary to initiate and maintain T-cell activity, and because signaling through the chimeric receptor alone is simply inadequate for T-cell maintenance or activation. The present invention identified an approach that solves this long-standing problem.

Although the present invention is directed to any antigen-specific T lymphocyte having an antigen specific-receptor which leads to increased in vivo survival of the T lymphocyte, in some preferred embodiments the T lymphocytes are specific for EBV. Over the past 6 years, Epstein-Barr virus-specific T cells have been safely and successfully infused into more than 150 patients (Khanna and Burrows, 2000; Heslop et al,. 1996; Rooney et al., 1995; Rooney et al., 1998). These T cells are readily manufactured ex vivo, and after infusion they expand and persist long-term, in a highly functional state. The difference in performance between EBV-specific and chimeric T cells can be explained as follows:

1) EBV infection is near universal and the virus and its antigens persist lifelong;

2) EBV infected B cells express antigens that can be recognized by T helper and T cytotoxic T cells on the same cell, allowing critical interactions to occur between these sub-populations of lymphocytes; and

3) EBV infected B cells express many different molecules that can co-stimulate T cells.

Expression of antitumor chimeric receptors on EBV-specific T lymphocytes provides a means of delivering effector cells that could persist in a functionally activated state due to their EBV specificity, and be capable of killing tumor cells through their chimeric receptor. This concept is also illustrated in FIG. 1.

In the Examples provided herein, EBV-specific T lymphocytes transduced with recombinant retrovirus encoding the G_{D2}-specific chimeric receptor 14.G2a-ζ efficiently recognized G_{D2} positive tumor cell targets, as demonstrated by tumor cell lysis and secretion of significant levels of IFN-γ, while maintaining specific and efficient HLA-restricted cytolysis of EBV-transformed cell lines. Both CD8⁺ and CD4⁺ CTL lines exerted tumor-specific cytotoxicity. Although lysis of the tumor target by gene-modified EBV-specific CTL was HLA independent, it could be inhibited by addition of non-labeled EBV target cells, while lysis of EBV positive targets could be diminished by competition from non-labeled tumor cells. EBV and G_{D2} negative cold targets had no effect. These results demonstrate functional dual specificity of transduced CTL for the native T cell receptor antigen and for the chimeric receptor target antigen.

In the Examples provided herein, EBV-specific T lymphocytes transduced with recombinant retrovirus encoding a CD19-specific chimeric receptor efficiently recognized CD19 positive tumor cell targets, as demonstrated by tumor cell lysis and secretion of significant levels of IFN-γ, while maintaining specific and efficient HLA-restricted cytolysis of EBV-transformed cell lines. Both CD8⁺ and CD4⁺ CTL lines exerted tumor-specific cytotoxicity. Although lysis of the tumor target by gene-modified EBV-specific CTL was HLA independent, it could be inhibited by addition of non-labeled EBV target cells, while lysis of EBV positive targets could be diminished by competition from non-labeled tumor cells. These results demonstrate functional dual specificity of transduced CTL for the native T cell receptor antigen and for the chimeric receptor target antigen.

Based on these in vitro and in vivo observations, in specific embodiments of the present invention EBV-specific T lymphocytes were transduced to express tumor-specific chimeric receptor genes and persist longer in vivo as functional anti-tumor cytotoxic effector cells than chimeric neuroblastoma cells generated from unselected peripheral blood T cells. Furthermore, the constant and powerful in vivo stimulus provided by presentation of EBV antigen in the context of appropriate costimulation is likely to prevent functional inactivation of chimeric receptor-transduced cells and enable them to maintain tumor-specific cytotoxicity. In some embodiments of the present invention, autologous EBV-specific T-lymphocytes that are transduced to express tumor-specific chimeric receptor genes are useful for adoptive immunotherapy of cancer, such as in a pediatric population. The near universality of EBV infection and the demonstrated safety and effectiveness of EBV-T cell infusions makes this “piggyback” approach a highly feasible strategy to augment any chimeric T cell immunotherapy for cancer or infection.

III. Chimeric Receptors

A. General Embodiments

In some embodiments of the present invention, a chimeric receptor is utilized to target and to activate T cells in a major histocompatibility complex-independent manner. In some embodiments, a single-chain fragment of variable regions (scFv) is used to create an antigen-binding domain on one polypeptide chain. In a specific embodiment, the scFv is derived from an antibody molecule by joining the VH and VL regions via a flexible peptide linker, which results in one continuous polypeptide molecule of the VL-linker-VH type or the VH-linker-VL type. The antigen-binding domain comprises an extracellular moiety of the chimeric receptor, which is combined with a transmembrane domain and an intracellular domain all within one polypeptide chain. In a preferred embodiment, the intracellular domain comprises a signaling domain, such as derived from the CD3-ζ chain of the TCR/CD3 complex or, in an alternative embodiment, derived from the high affinity IgGεFc receptor g-chain (FcεRIγ). Thus, a variety of chimeric single-chain receptors which endow T cells with MHC-independent specificity to various cells, such as tumor cells, may be generated.

A skilled artisan recognizes that at least three characteristics are desirable for a chimeric receptor comprised on one contiguous polypeptide chain to demonstrate both antibody-like specificity and cellular activation capacity, including: 1) a single-chain binding domain with specificity for a particular membrane-bound antigen; 2) receptor-mediated cellular activation; and 3) stable expression of the receptor on the T cell surface.

A skilled artisan recognizes that phage display techniques are useful for rapid and efficient generation of single-chain antigen-binding domains derived from monoclonal antibodies. Methods to enrich recombinant phages that express scFv with specificity to membrane-bound antigens are known (Hombach et al., 1998)

Chimeric receptors utilized in the present invention may be constructed from cDNAs encoding the desired segments, although other methods are readily apparent to those of ordinary skill in the art. In one method, for example, the chimeric receptor DNA is prepared by providing cloned cDNAs encoding an extracellular region from a selected receptor and transmembrane and cytoplasmic domains. These cloned cDNAs, if prepared by restriction enzyme digestion, may contain unwanted sequences that would intervene in the fusion. The unwanted sequences are removable by techniques known to those of ordinary skill in the art, including loop-out site-directed mutagenesis or splice-overlap extension polymerase chain reaction (PCR). The sequence of the chimeric cDNA encoding the receptor may then be confirmed by standard DNA sequencing methods. Specific examples of such chimeric receptors are illustrated in Specific Embodiments.

The polynucleotide regions encoding the chimeric receptors are generally operably linked to control regions that allow expression of the chimeric receptor in a host cell, particularly a CTL. Control regions include, at least, a promoter and a ribosomal binding site, and may also include, inter alia, enhancer regions, splice regions, polyadenylation regions, transcription and/or translation termination regions, and transcription and/or translation factor binding sites. These control regions may be present in recombinant vectors, particularly in recombinant expression vectors.

The ability of the chimeric receptor to enhance activation and proliferation of the host CTL is readily demonstrated by techniques known in the art. For example, cell lines that express the chimeric receptors can be stimulated via the TCR pathway by providing any of a variety of means for stimulating the TCR, and then tested for activation and proliferation in the absence of cytokines that are normally required for growth of the CTL.

Techniques for nucleic acid manipulation are described generally, for example, in Sambrook et al. (1989), Ausubel et al. (1987), and in Annual Reviews of Biochemistry (1992) 61:131-156. Reagents useful in applying such techniques, such as restriction enzymes and the like, are widely known in the art and commercially available from a number of vendors.

Large amounts of the polynucleotides used to create the cells of the present invention may be produced by replication in a suitable host cell. The natural or synthetic polynucleotide fragments coding for a desired fragment may be incorporated into recombinant nucleic acid constructs, typically polynucleotide constructs, capable of introduction into and replication in a prokaryotic or eukaryotic cell. Usually the constructs will be suitable for replication in a unicellular host, such as yeast or bacteria, but may also be intended for introduction to, with and without and integration within the genome, cultured mammalian or plant or other eukaryotic cell lines. Purification of nucleic acids produced by the methods of the present invention can be achieved by methods known in the art and described, e.g., in Sambrook et al. (1989) and Ausubel et al. (1987). Of course, the polynucleotides used in the present invention may also be produced in part or in total by chemical synthesis, e.g., by the phosphoramidite method described by Beaucage and Carruthers (1981) Tetra. Letts. 22:1859-1862 or the triester method according to Matteucci et al. (1981) J. Am. Chem. Soc. 103:3185, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

Polynucleotide constructs prepared for introduction into a prokaryotic or eukaryotic host cell for replication will typically comprise a replication system recognized by the host, including the intended recombinant polynucleotide fragment encoding the desired polypeptide. Such vectors may be prepared by means of standard recombinant techniques well known in the art and discussed, for example, in Sambrook et al. (1989) or Ausubel et al.

Preferably, the polynucleotide construct will contain a selectable marker, a gene encoding a protein necessary for the survival or growth of a host cell transformed with the vector. The presence of this gene ensures the growth of only those host cells which express the inserts. Typical selection genes encode proteins that (a) confer resistance to antibiotics or other toxic substances, e.g. ampicillin, neomycin, methotrexate, etc.; (b) complement auxotrophic deficiencies, or (c) supply critical nutrients not available from complex media, e.g. the gene encoding D-alanine racemase for Bacilli. The choice of the proper selectable marker will depend on the host cell, and appropriate markers for different hosts are well known in the art.

The polynucleotides of the present invention may be introduced into the desired T cell by any of a variety of means known in the art, including, for example, transformation, electroporation, lipofection, and transduction, including the use of viral vectors, which are currently a preferred means of introduction, as described below.

Various infection techniques have been developed which utilize recombinant infectious virus particles for gene delivery. This represents a preferred approach to the present invention. The viral vectors which have been used in this way include virus vectors derived from simian virus 40 (SV40) (Karlsson et al., Proc. Natl. Acad. Sci. USA 84 82:158, 1985); adenoviruses (Karlsson et al., EMBO J. 5:2377, 1986); adeno-associated virus (AAV) (B. J. Carter, Current Opinion in Biotechnology 1992, 3:533-539; and Flotte et al., U.S. patent application Ser. No. 08/149,332, abandoned, filed Nov. 9, 1993); and retroviruses (Coffin, 1985, pp. 17-71 in Weiss et al. (eds.), RNA Tumor Viruses, 2nd ed., Vol. 2, Cold Spring Harbor Laboratory, New York). Thus, gene transfer and expression methods are numerous but essentially function to introduce and express genetic material in mammalian cells. Several of the above techniques have been used to transduce hematopoietic or lymphoid cells, including calcium phosphate transfection (Berman et al., 1984), protoplast fusion (Deans et al., 1984), electroporation (Cann et al., Oncogene 3:123, 1988), and infection with recombinant adenovirus (Karlsson et al.; Reuther et al., Mol. Cell. Biol. 6:123, 1986), adeno-associated virus (LaFace et al., 1988) and retrovirus vectors (Overell et al., Oncogene 4:1425, 1989). Primary T lymphocytes have been successfully transduced by electroporation (Cann et al., supra, 1988) and by retroviral infection (Nishihara et al., Cancer Res. 48:4730, 1988; Kasid et al., 1990; and Riddell, S. et al., Human Gene Therapy 3:319-338, 1992).

B. Specific Embodiments

A skilled artisan recognizes that there are a multitude of chimeric receptors known and used in the art. Specific examples include:

14.G2a-ζ (Target antigen: GD2, expressed on neuroblastomas)

CD19-ζ (Target antigen: CD19, expressed on leukemic blast cells)

CD20-ζ (Target antigen: CD20, expressed on leukemia blast cells and lymphoma cells)

ETAA16-ζ (Target antigen: unidentified structure on Ewing tumors)

In addition, single-chain antibodies used as the recognition portion of chimeric receptors include:

scFv 763.74, anti-HMW MAA (melanoma); Hombach et al. (1999); Abken et al. (2001)

scFv anti-Neu/HER2 (ovarian cancer); Stancovski et al. (1993)

scFv MOv18, anti-FRP (ovarian cancer); Hwu et al. (1995)

scFv (G250) (renal cell carcinoma); Weijtens et al. (1996; 1998; 2000)

scFv (B72.3), anti-TAG72 (colon cancer); Hombach et al. (1997)

scFv (MFE) anti-CEA (colon cancer); Darcy et al. (1998)

scFv (F11-39) anti-CEA (colon cancer); Kuroki et al. (2000)

scFv CC49, anti-TAG-72 (colon cancer); McGuinness et al. (1999)

scFv GA733.2 (colon cancer); Ren-Heidenreich et al. (2000)

scFv (HRS3), anti-CD30 (Hodgkin's lymphoma); Hombach et al. (1998)

scFv FRP5, anti-huErbB-2 (ovarian cancer); Moritz et al. (1994); Moritz and Groner, 1995, Altenschmidt et al. (1996; 1997)

scFv 3G6, anti-G_{D}2 (neuroblastoma); Krause et al. (1998)

CD4, EC domain (high affinity receptor for HIV gp120); Romeo and Seed, 1991; Roberts et al. (1994; 1998); Tran et al. (1995); Hege et al. (1996), Yang et al. (1997)

scFv b12 (anti-HIV gp120 cnv gp); Bitton et al. (1998)

Anti-collagenase type II scFv (rheumatoid arthritis); Annenkov et al. (1998)

Thus, a skilled artisan recognizes that in addition to targeting cancer, chimeric receptors have been designed for treating other chronic diseases, including infectious (HIV) and autoimmune (rheumatoid arthritis) diseases.

A skilled artisan recognizes that in the embodiments wherein an antitumor chimeric receptor is utilized, the tumor may be of any kind as long as it has a cell surface antigen which may be recognized by the chimeric receptor. In a specific embodiment, the chimeric receptor may be for any cancer for which a specific monoclonal antibody exists or is capable of being generated. In particular, cancers such as neuroblastoma, small cell lung cancer, melanoma, ovarian cancer, renal cell carcinoma, colon cancer, Hodgkin's lymphoma, and childhood acute lymphoblastic leukemia have antigens specific for the chimeric receptors.

IV. Enhancement of an Immune Response

Immunotherapeutics, generally, rely on the use of immune effector cells and molecules to target and destroy cancer cells. The effector may be a lymphocyte carrying a surface molecule that interacts, either directly or indirectly, with a tumor cell target. Various effector cells include cytotoxic T cells and NK cells. In some embodiments of the present invention, the methods and compositions described herein are utilized in conjunction with another type of therapy for cancer, such as chemotherapy, surgery, radiation, gene therapy, and so forth.

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes genetically modified to express a tumor-specific chimeric receptor gene as described herein. The activated lymphocytes will most preferably be the patient's own cells that were earlier isolated from a blood or tumor sample and activated and expanded in vitro.

The present invention includes a method of enhancing the immune response in a subject comprising the steps of contacting one or more EBV-specific T lymphocytes with a receptor protein composition, such as by transducing the cell with a vector comprising a chimeric receptor. As used herein, a “receptor protein composition” may comprise a chimeric receptor (e.g., a peptide or polypeptide) or a nucleic acid encoding a chimeric receptor (e.g., a chimeric receptor expression vector.

In certain embodiments, the one or more lymphocytes is comprised in an animal, such as a human. In certain embodiments, the animal is a human cancer patient. In a preferred aspect, the one or more lymphocytes comprise a T-lymphocyte. In a particularly preferred embodiment, the T-lymphocyte is a cytotoxic T-lymphocyte.

The present invention regards an adoptive immunotherapy approach in which lymphocyte(s) are obtained from an animal (e.g., a patient previously exposed to an antigen, such as Epstein-Barr virus) and comprising antigen-specific T lymphocytes). These T lymphocytes are transduced with composition comprising a chimeric receptor, preferably a nucleic acid encoding a chimeric receptor. In a specific embodiment, the lymphocyte may comprise an additional immunostimulatory agent or a nucleic acid encoding such an agent. The lymphocyte(s) may be obtained, for example, from the blood of the subject. In certain preferred embodiments, the lymphocyte(s) are peripheral blood lymphocyte(s). In a preferred embodiment, the lymphocyte(s) are administered to the same or different animal (e.g., same or different donors). In a preferred embodiment, the animal (e.g., a patient) has or is suspected of having a cancer, such as for example, breast cancer, prostate cancer, neuroblastoma, small cell lung cancer, melanoma, ovarian cancer, renal cell carcinoma, colon cancer, Hodgkin's lymphoma, or childhood acute lymphoblastic leukemia. In other embodiments, the method of enhancing the immune response is practiced in conjunction with a cancer therapy.

In certain embodiments, EBV-specific T-lymphocytes are specifically contacted with an antigenic composition of the present invention, such as a nucleic acid encoding a chimeric receptor. In general, T cells express a unique antigen binding receptor on their membrane (T-cell receptor), which can only recognize antigen in association with major histocompatibility complex (MHC) molecules on the surface of other cells. A skilled artisan recognizes that generally there are several populations of T cells, such as T helper cells and T cytotoxic cells. T helper cells and T cytotoxic cells are primarily distinguished by their display of the membrane bound glycoproteins CD4 and CD8, respectively. T helper cells secrete various lymphokines that are crucial for the activation of B cells, T cytotoxic cells, macrophages and other cells of the immune system. In contrast, a T cytotoxic cell that recognizes an antigen-MHC complex proliferates and differentiates into an effector cell called a cytotoxic T lymphocyte (CTL). CTLs eliminate cells of the body displaying antigen, such as virus infected cells and tumor cells, by producing substances that result in cell lysis.

CTL activity can be assessed by methods described herein or as would be known to one of skill in the art. For example, CTLs may be assessed in freshly isolated peripheral blood mononuclear cells (PBMC), in a phytohaemagglutinin-stimulated IL-2 expanded cell line established from PBMC (Bernard et al., 1998) or by T cells isolated from a previously immunized subject and restimulated for 6 days with DC infected with an adenovirus vector containing antigen using standard 4 h ⁵¹Cr release microtoxicity assays. One type of assay uses cloned T-cells. Cloned T-cells have been tested for their ability to mediate both perforin and Fas ligand-dependent killing in redirected cytotoxicity assays (Simpson et al., 1998). The cloned cytotoxic T lymphocytes displayed both Fas- and perforin-dependent killing. Recently, an in vitro dehydrogenase release assay has been developed that takes advantage of a new fluorescent amplification system (Page et al., 1998). This approach is sensitive, rapid, reproducible and may be used advantageously for mixed lymphocyte reaction (MLR). It may easily be further automated for large scale cytotoxicity testing using cell membrane integrity, and is thus considered in the present invention. In another fluorometric assay developed for detecting cell-mediated cytotoxicity, the fluorophore used is the non-toxic molecule alamarBlue (Nociari et al., 1998). The alamarBlue is fluorescently quenched (i.e., low quantum yield) until mitochondrial reduction occurs, which then results in a dramatic increase in the alamarBlue fluorescence intensity (i.e., increase in the quantum yield). This assay is reported to be extremely sensitive, specific and requires a significantly lower number of effector cells than the standard ⁵¹Cr release assay.

In certain aspects, T helper cell responses can be measured by in vitro or in vivo assay with peptides, polypeptides or proteins. In vitro assays include measurement of a specific cytokine release by enzyme, radioisotope, chromophore or fluorescent assays. In vivo assays include delayed type hypersensitivity responses called skin tests, as would be known to one of ordinary skill in the art.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Cell Lines and Antibodies

The neuroblastoma cell line LAN-1 was provided by Dr. Seeger's laboratory at UCLA, and the JF line was established by in vitro cultivating primary tumor cell suspensions obtained from a child with neuroblastoma for multiple passages. The ecotropic packaging cell line Phoenix (Kinsella et al., 1996) was provided by Gary P. Nolan, Stanford, Calif. A-204, HSB-2, Jurkat and PG-13 cells were obtained from American Type Culture Collection. The hybridoma cell line 14.G2a (mouse IgG2a;K) (Mujoo et al., 1989) was provided by Ralph A. Reisfeld (La Jolla, Calif.), and anti-14.G2a idiotypic antibody 1A7 (Sen et al., 1997) (TriGem) by Titan Pharmaceuticals Inc., South San Francisco. The LMP-2/HLA-A2 tetramer (HLA-A-0201/CLGGLLTMV) was obtained from the MHC Tetramer Core Facility (Atlanta, Ga.).

### Example 2

### Construction of Chimeric Receptor Genes

The variable domains of monoclonal antibody 14.G2a were cloned as single-chain Fv (scFv) molecules into the replicative form of fUSE5 vector phage DNA (Smith, 1985), G_{D2}-binding phage were selected by immunoscreening with ELISA. Chimeric γ chain receptor genes were assembled using pRSV-γ1. The human ζ chain transmembrane and cytoplasmic portions were amplified from pGEM3ζ (Weissman et al., 1988). A truncated receptor was engineered by PCR by inserting a stop codon after the first three cytoplasmic amino acids. The chimeric genes were subcloned into the BamHI and NcoI sites of the retroviral vector SFG (Riviere et al., 1995) (provided by R. C. Mulligan, Cambridge, Mass.).

### Example 3

### Production of Recombinant Retrovirus

Fresh retroviral supernatants collected from transiently transfected Phoenix-eco cells were used to infect the packaging cell line PG13 in the presence of polybrene (8 μg/ml) twice for 48 hours at 32° C. Viral supernatants were generated on the resulting bulk producer cell lines for 24 hours at 32° C.

### Example 4

### Generation of EBV-Transformed B Cell Lines

Peripheral blood-derived mononuclear cells (5×10⁶) were incubated with 10 μl of concentrated supernatant from the EBV producer cell line B95-8 in a total of 200 μl medium for 30 min. The cells were then plated at 10⁶ cells per well in a flat-bottomed 96-well plate in RPMI 1640 medium (GIBCO-BRL, Gaithersburg, Md.) containing 10% FCS (Hyclone, Logan, Utah), and 2 mM L-glutamine (Biowhittaker, Walkersville, Md.), as well as 1 μg/ml of cyclosporin A (Sandoz Pharmaceuticals, Washington D.C.). Cells were fed weekly until lymphoblastoid cell lines were established.

### Example 5

### Generation and Transduction of EBV-Specific CTL Cultures

Peripheral blood-derived mononuclear cells (2×10⁶) were cocultured with 5×10⁴ γ-irradiated (40 Gy) autologous LCLs per well in a 24-well plate. Starting on day 10, the responder cells were restimulated weekly with irradiated LCLs at a responder:stimulator ratio of 4:1. Two weekly doses of rhIL-2 (40 IU/ml) were added from day 14. Twenty-four hours following the third stimulation, the cells were transferred to a 24-well plate precoated with OKT-3 (1 μg/ml; Ortho Pharmaceuticals, Raritan, N.J.) and anti-CD28 antibody (1 μg/ml; Pharmingen, San Diego, Calif.) at 1×10⁶ cells per well and incubated for 48 hours. Cells were transduced in 24-well plates (Becton Dickinson, Franklin Lakes, N.J.), coated with recombinant FN CH-296 (Retronectin, Takara Shuzo, Otsu, Japan) at a concentration of 4 μg/cm. The prestimulated CTLs were resuspended at 1×10⁶ cells/ml in culture medium containing rhIL-2 (100 IU/ml), and incubated with equal volumes of freshly generated viral supernatant for 36 hours at 37° C. and 5% CO₂.

### Example 6

### Flow Cytometry

Cells were stained with fluorescein-conjugated monoclonal antibodies (Becton Dickinson, San Jose, Calif.) directed against CD3, CD4, CD8, CD16, CD56 and CD25 surface proteins. For each sample, 10,000 cells were analyzed by FACSCalibur with the Cell Quest Software (Becton Dickinson, San Jose, Calif.). Surface expression of 14.G2a-ζ was analyzed after incubation of CTL (1×10⁶) with 14.G2a anti-idiotypic antibody 1A7 (200 ng/5×10⁵ cells) in the presence of normal goat serum for 20 min on ice, followed by incubation with fluorescein isothiocyanate (FITC)-labeled goat antimouse antibody (Becton Dickinson, San Jose, Calif.). For tetramer staining, CTLs from HLA-A2⁺ donors were incubated with phycoerythrin (PE)-labeled LMP-2/HLA-A2 tetramer at a final concentration of 50 mg/ml in PBS+2% FCS for 30 min on ice, then washed and stained with PerCP-labeled anti-CD8 antibody for 20 min. One million events were acquired and analyzed.

### Example 7

### Measurement of Cytokine Production

Duplicate samples of transduced effector cells (5×10⁴/well) were cocultured with various tumor cells or EBV-transformed LCL at target-to-effector ratios of 3:1 and 1:3 in 96-well round bottomed plates. After 24 hours, the supernatants were harvested and analyzed for human IFN-γ, TNF-α, IL-4, IL-10 and IL-12 (Pharmingen, San Diego, Calif.) or GM-CSF (R&D Systems, Minneapolis, Minn.) by ELISA according to the manufacturer's instructions.

### Example 8

### Cytotoxicity Assays

Cytotoxic specificity was determined in standard ⁵¹Cr release assays. Various numbers of T effector cells were coincubated in triplicate with 5000 target cells labeled with 100 μCi ⁵¹ Cr/0.5×10⁶ cells in a total volume of 200 μl in a V-bottomed 96-well plate. At the end of a 4-hour period at 37° C. and 5% CO₂, supernatants were harvested, and radioactivity was counted in a gamma counter. Maximum release was determined by lysis of target cells with Triton X. To determine HLA class I or II restriction of cytolysis, target cells were preincubated for 30 min with 16.5 ng/ml of W6/32 or CR3/43 antibodies (Dako, Carpinteria, Calif.). For cold target inhibition assays, unlabeled inhibitor cells (cold targets) were seeded in plates at various cold-to-hot target ratios. Effector cells were then added and incubated for 30 min at 37° C. before labeled target cells (hot targets) were added.

### Example 9

### Proliferation Assays

Transduced T lymphocytes were coincubated in triplicate at 5×10⁴ cells/well with various tumor cell targets or autologous or allogeneic EBV-LCL at a 4:1 stimulator to responder ratio. Following a 72-hr coincubation period, wells were pulsed with 2.5 μCi of [³H] thymidine for 18 hr, and the samples were harvested onto glass fiber filter paper for β scintillation counting.

### Example 10

### Transduced EBV-Specific CTLs Express the Chimeric Receptor While Maintaining Their Immunophenotype

Eight EBV-specific CTL lines, generated from four different seropositive healthy donors (Rooney et al., 1995; Rooney et al., 1998), were transduced with 14.G2a-ζ chimeric receptor genes. This receptor is derived from the 14.G2a monoclonal antibody which recognizes G_{D2}, a ganglioside antigen present on tumors of neural crest origin (Mujoo et al., 1989; Schulz et al., 1984), including neuroblastoma and small cell lung cancer, as well as glioblastoma and melanoma.

Cells of a representative EBV-specific CTL line, 5 days after retroviral transduction with 14.G2a-ζ chimeric receptor genes, were stained with 14.G2a idiotype specific monoclonal antibody 1A7, followed by incubation with FITC-labeled goat antimouse antibody, and then peridinin chlorophyll protein (PerCP)-labeled anti-CD8 or anti-CD4 antibody. Surface immunofluorescence was analyzed by flow cytometry.

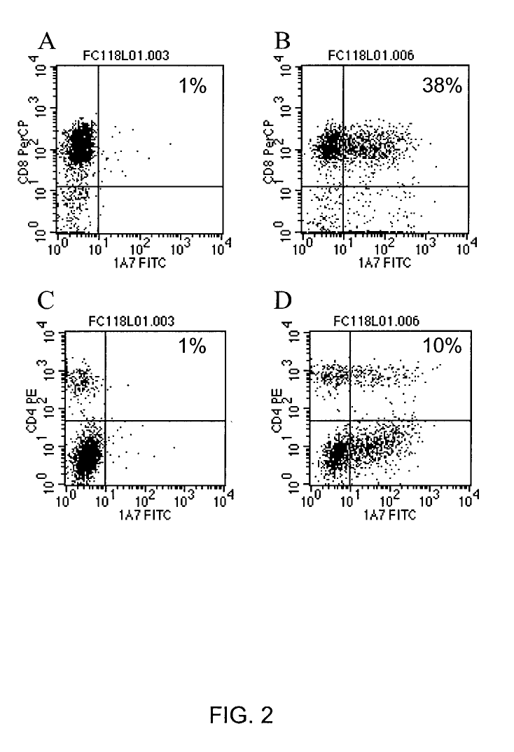

Flow cytometric analysis of CTLs stained with anti-14.G2a idiotype-specific antibody identified chimeric receptors on 10.2-43.1% of the CTLs (mean, 16.5%). Chimeric receptor expression was maintained over the entire period of culture (up to 45 days) without any apparent downregulation. CD4⁺ and CD8⁺ T lymphocytes within the cultured cell population were transduced equally well (FIG. 2).

After three stimulations with autologous LCLs, the majority of the CTL lines had a characteristic immunopheno type 12-15: CD3⁺ CD8⁺ on 80>90% of the cells and CD3⁺ CD4⁺ (T cell helper) on 4-21%. Fewer than 5% of the cells showed an immunophenotype characteristic of NK cells (CD3⁻ CD16⁺ CD56⁺). One cell line was predominantly CD3⁺ CD4⁺. Transduction of CTLs did not result in any changes of cellular immunophenotypes by comparison with nontransduced cells. Hence, introduction and expression of the chimeric receptor does not shift the phenotype of EBV-specific CTLs from that known to be able to expand, persist and induce tumor regression following infusion in vivo (Heslop et al., 1996; Roskrow et al., 1998; Rooney et al., 1995; Rooney et al., 1998).

### Example 11

### Triggering of the Native TCR but not The ζ Chimera Induces Proliferation and Expansion of Modified EBV-Specific CTLs

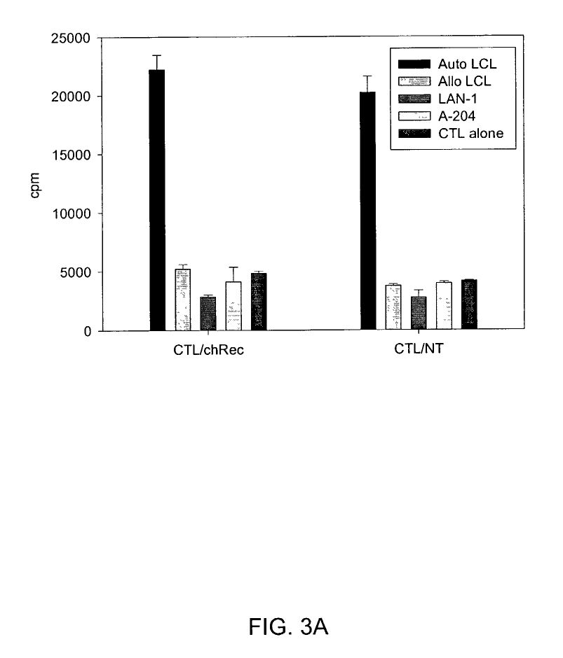

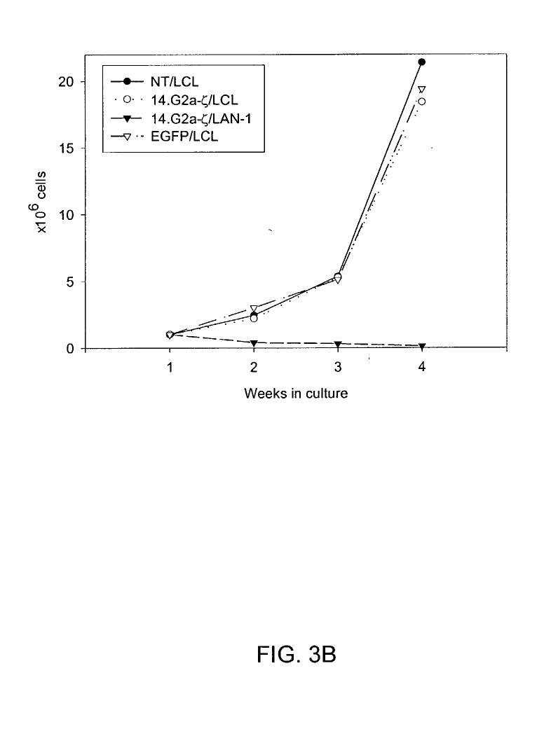

To confirm that signaling through the chimeric receptor alone provides a proliferation stimulus insufficient for in vitro expansion of the cells, 14.G2a-ζ transduced and nontransduced EBV-CTLs were cultured in the presence of LCLs, irradiated G_{D2}⁺ (LAN-2) and G_{D2}⁻ (A-204) tumor cell targets and rhIL-2 (40 IU/ml), or EBV-CTLs alone. Incubation with the tumor targets did not elicit a proliferative response from either the modified or unmodified cells, as demonstrated by [³H]thymidine incorporation (FIG. 3a). In contrast, autologous LCLs triggered substantial [³H] thymidine uptake by both types of CTL lines. Furthermore, whereas transduced CTL continued to expand in response to stimulation with irradiated autologous LCLs, with kinetics similar to those of nontransduced cells, the transduced CTL could not be maintained in culture for longer than 4 weeks when stimulated with tumor cells alone (FIG. 3b). These results are compatible with in vivo data showing that chimeric receptor stimulation alone is inadequate to maintain T cell proliferation and expansion (Brocker and Karjalainen, 1995; Brocker, 2000).

### Example 12

### Coculture with Tumor Cells Does Not Result in CTL Lysis or Growth Inhibition

To exclude the possibility that coculture with G_{D2}⁺ tumor cells is toxic to T cells independent of chimeric receptor triggering, control experiments were performed by testing the cytotoxicity of G_{D2}⁺ tumor cells towards chimeric receptor-transduced CTLs in standard ⁵¹Cr release assays, and by comparing the expansion of CTLs in the presence of EBV and tumor targets.

EBV-specific CTL were not susceptible to cytolysis by JF or LAN-1 tumor cells following coincubation periods of up to 18 hours. Furthermore, CTL expansion was not decreased in the presence of tumor cells, when compared to allogeneic LCL or absence of stimulator cells, indicating that the tumor cells did not actively inhibit CTL expansion.

### Example 13

### Chimeric Receptor-Modified CTL Specifically Release Cytokines in Response to Autologous EBV and G_{D2}⁺ Tumor Targets

Having demonstrated that chimeric EBV specific T cells could be readily expanded ex vivo, functional activation of transduced EBV-specific CTL by their native T cell receptor and chimeric receptor-defined cellular targets was compared. The pattern of cytokine release by modified T lymphocytes after incubation with G_{D2}⁺ and G_{D2}⁻ tumor target cells was determined, as well as with autologous EBV-infected LCLs.

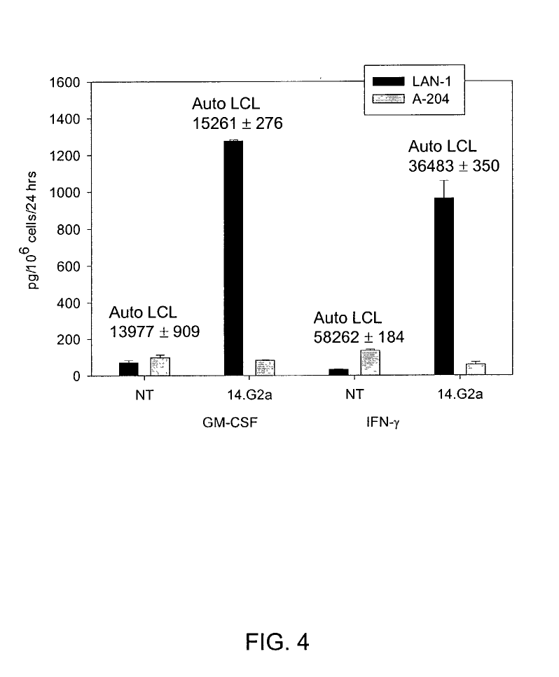

CTL cultures containing 5-15% 14.G2a-ζ transduced T lymphocytes released IFN-γ (up to 2982 pg/m1×10⁶ cells/24 hr) and GM-CSF (up to 1278 pg/m1×10⁶ cells/24 hr), as well as trace amounts of TNF-α upon stimulation with G_{D2}⁺ target cells in the absence of specific cytokine release during incubation with G_{D2}⁻ tumor cell targets (FIG. 4). Nontransduced CTLs and cell lines transduced with the truncated chimeric receptor variant 14.G2a-ζ did not release cytokines in response to incubation with G_{D2}⁺ tumor targets. IL-4, IL-10 and IL-12 were not detected in the supernatants of the stimulated cells. A similar pattern of cytokine release was observed when cells were cultured with autologous EBV-LCLs. No differences in the cytokine response to EBV targets were found between nontransduced and transduced CTL. Nonetheless, the quantity of specific IFN-γ and GM-CSF release by CTLs in response to autologous LCL targets exceeded the cytokine release triggered by stimulation of transduced CTL populations with G_{D2}⁺ tumor targets by 8-49-fold (FIG. 4). Hence, while chimeric receptor-positive EBV-infected CTLs retain the capacity to respond to stimulation of the native receptor, chimeric receptor engagement results in comparatively low levels of specific IFN-γ and GM-CSF secretion.

### Example 14

### Chimeric Receptor-Modified CTLs Specifically Lyse Autologous EBV and GD2+ Tumor Targets

The cytolytic specificities of nontransduced and transduced cells were compared in standard 4-hr ⁵¹Cr release assays (Table 1).

TABLE 1
Cytolytic characteristics of EBV-specific
CTLs with or without chimeric
receptors.

| | Percent specific lysis^{a} | | | | | | | Day |
|---|---|---|---|---|---|---|---|---|
| | Nontransduced | | | 14.G2a-ζ transduced | | | | post- |
| | Auto | Allo | LAN- | Auto | Allo | LAN- | | trans- |
| CTL line | LCL | LCL | 1 | LCL | LCL | 1 | A-204 | duction |
| 1 | 61 | 23 | 0 | 55 | 19 | 45 | | 29 |
| 2 | 49 | 5 | 0 | 48 | 5 | 47 | | 8 |
| 3 | 49 | 11 | 0 | 38 | 5 | 26 | | 8 |
| 4 | 53 | 12 | | 35 | 13 | 50 | | 8 |
| 5 | 50 | 5 | | 42 | 3 | 21 | | 7 |
| 6 | 49 | 22 | | 66 | | 28 | | 22 |
| 7 | 33 | 6 | | 30 | 8 | 48 | 0 | 9 |
| 8 | 27 | 7 | | 52 | 3 | 43 | 5 | 9 |
| 9 | 45 | 0 | 5 | 45 | 5 | 32 | 4 | 9 |
| 10^{r} | 81 | 10 | 1 | 65 | 0 | 26 | 0 | 15 |

^{a} = E:T ratio 40:1
^{r} = CTL line 5, retested after additional restimulation with autologous EBV-LCL

Following transduction, the CTLs had received at least one and up to four further weekly stimulations with autologous EBV targets before cytotoxic activity was assessed. The corresponding nontransduced CTLs had received an equal number of restimulations. At an effector-target ratio of 40:1, the percentage of autologous LCL targets lysed by nontransduced CTL lines ranged from 27% to 81% (mean, 49.6%), including one CTL line with a high percentage of CD4⁺ T cells. After transduction with chimeric receptor genes, 30-66% (mean, 47.6%) of autologous LCL targets were lysed by these lines. Under the same conditions, only 5-23% (mean, 10.1%) of HLA-mismatched EBV-transformed targets were lysed by nontransduced CTLs, compared with 3-19% (mean, 6,7%) by 14.G2a-ζ-transduced cells. None of the lines had discernible activity against autologous phytohemagglutinin-stimulated lymphoblasts. Whereas nontransduced CTL were incapable of lysing tumor targets (0-5%), 21-50% (mean, 36,6%) of G_{D2}⁺ LAN-1 tumor cells were lysed during coincubation with 14.G2a-ζ transduced CTL. No cytolytic activity of transduced cells against the G_{D2}⁻ tumor target A-204 was seen. The specificity of the interaction with the G_{D}₂-expressing tumor cell targets was further confirmed by preincubation of G_{D2}-positive target cells with 14.G2a mAb, resulting in up to 81% inhibition of lysis by 14.G2a-ζ transduced cells. The cytolytic activity of cell lines tested for up to 29 days post-transduction (four stimulations) was comparable to that of lines tested over shorter intervals. Retesting of a CTL line after an additional round of restimulation with autologous LCL resulted in comparable cytolytic activity against both EBV and tumor targets (Table 1, CTL lines #5 and #10).

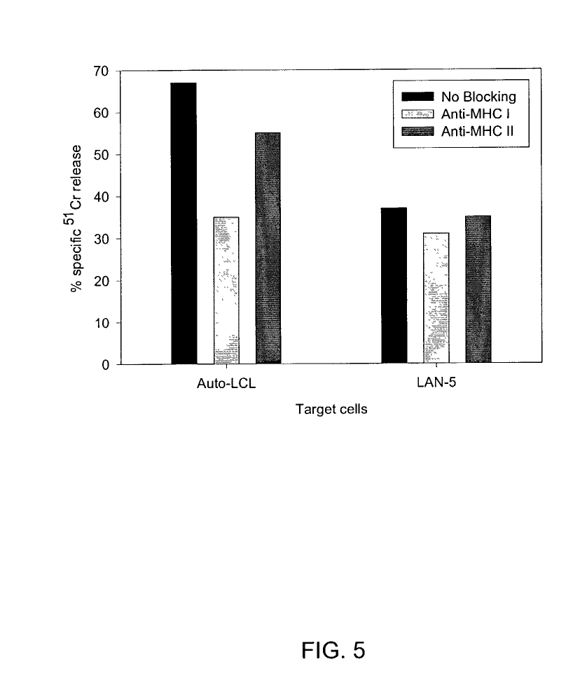

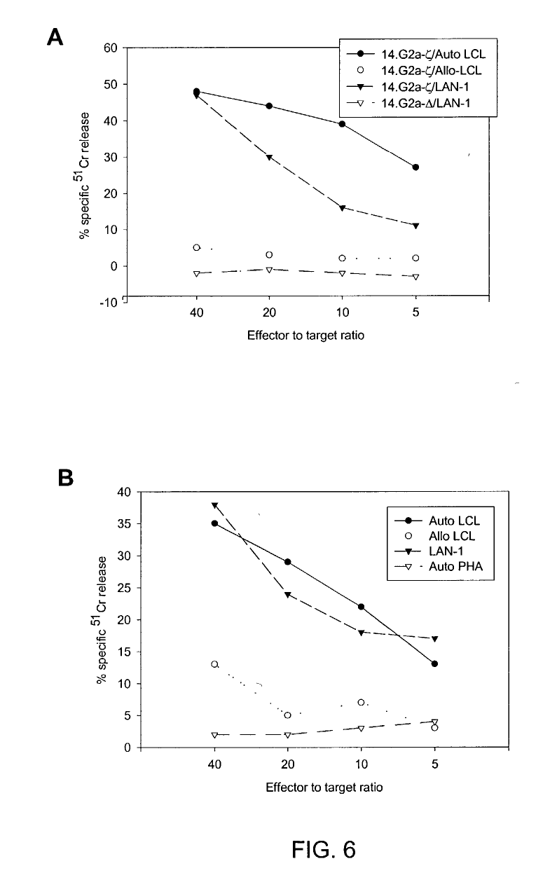

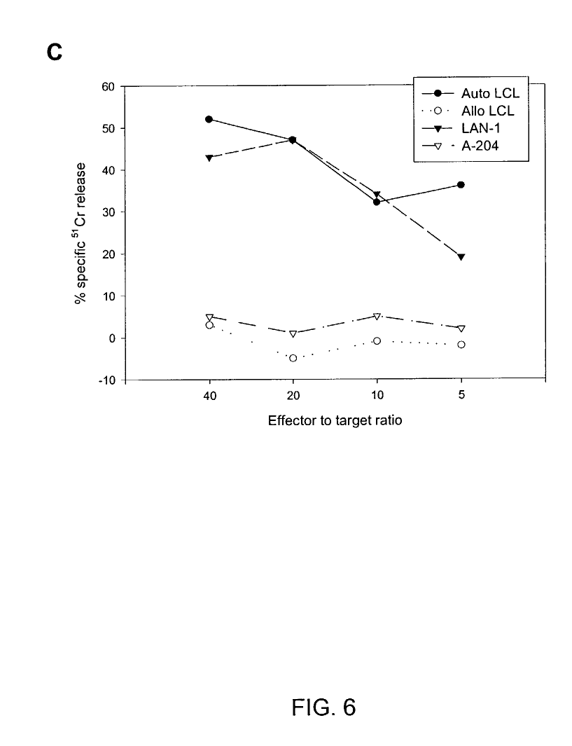

FIG. 5 compares the cytolytic activities of three different 14.2a-ζ-transduced CTL lines against EBV and tumor cell targets. The chimeric receptor-bearing T cells recognized and lysed G_{D2}⁺ tumor cells (LAN-1) and autologous LCLs with similar efficiency over the entire range of effector-to-target ratios, but responded poorly to G_{D2}⁻ tumor cells (A-204). Inhibition studies with anti-MHC antibodies identified MHC class I as the major restriction element for LCL lysis by nontransduced and 14.G2a-ζ transduced CD8⁺ CTL lines. However, preincubation with HLA class I and II blocking antibodies did not affect the lysis of LAN-1 target cells, indicating a lack of MHC restriction (FIG. 6). These results establish that the EBV-specific chimeric T cells kill their EBV targets through their (MHC-restricted) conventional receptor, and their tumor targets through the (MHC-unrestricted) chimeric receptor.

### Example 15

### 14.G2A-ζ-Transduced EBV-Specific CTLs have Functional Specificity for Both EBV and Tumor Antigens

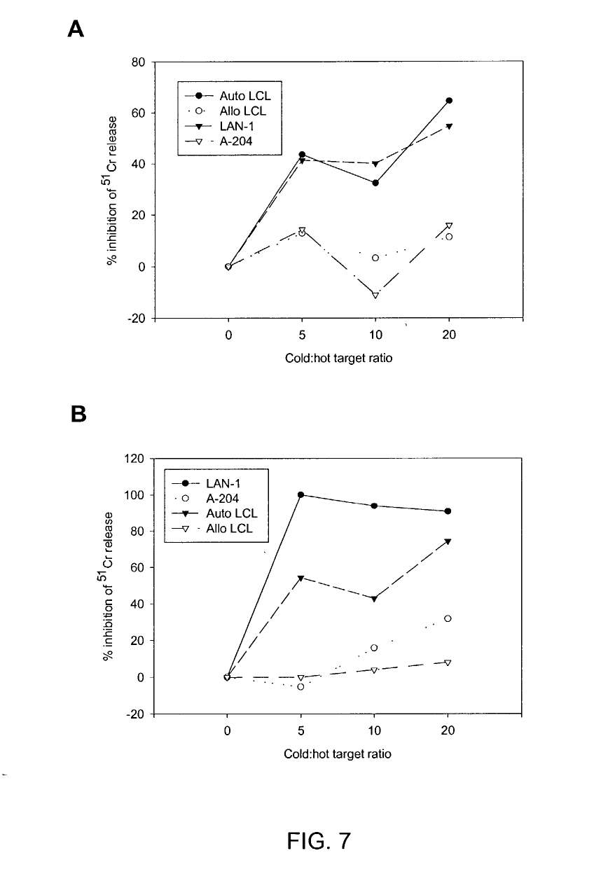

Because of the superior in vivo functional capabilities of EBV-specific cell lines over single epitope specific clones (Walter et al., 1995; Gottschalk et al., 2001), the preceding studies were performed on mixed populations of cytotoxic T lymphocytes. Therefore to demonstrate that transduced populations of EBV-specific CTLs are functionally bispecific for the native T cell receptor antigen and the chimeric receptor target, rather than recognizing such targets independently of one another, cold target inhibition assays were performed. The antigen specificity of both tumor cell and LCL lysis by gene-modified EBV-specific CTL lines was determined by analyzing the capacity of unlabeled autologous LCLs and G_{D2}⁺ tumor cells to block lysis of tumor cells and LCLs, respectively. Although lysis of the G_{D2}⁺ tumor target LAN-1 by 14.G2a-ζ-transduced EBV-specific CTLs was HLA-independent, it could be inhibited by addition of nonlabeled EBV target cells, while lysis of EBV+ targets could be diminished by competition from nonlabeled tumor cells (FIG. 7). Neither allogeneic EBV-LCL nor G_{D2}⁻ cold targets had an effect on the lysis of autologous EBV-LCL and G_{D2}⁺ hot targets.

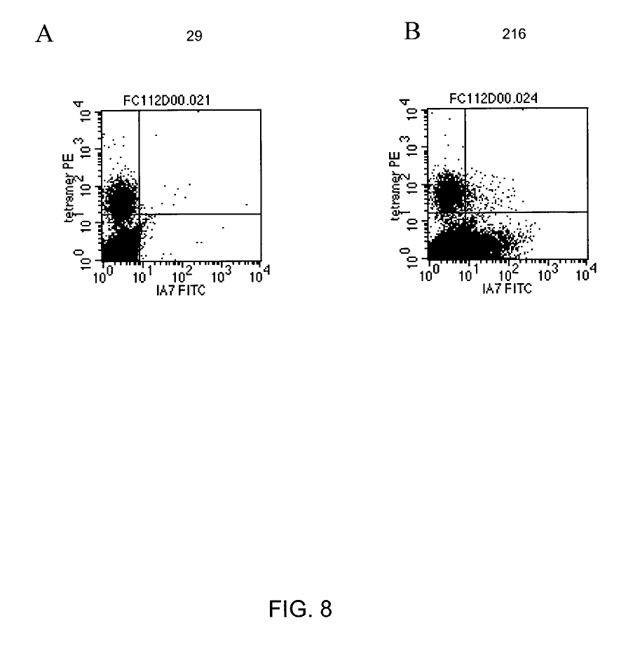

As an additional demonstration of coexpression of the T cell receptor, specific for an EBV-encoded antigen, as well as the 14.G2a-ζ chimeric receptor on individual cells within the CTL culture population, a transduced CTL line from an HLA-A2⁺ donor was stained with LMP-2 specific tetramers and with anti-14.G2a-idiotype antibody 1A7 (FIG. 8). Slightly more than 7% of the cells specific for LMP-2 had detectable levels of 14.G2a-ζ surface expression, comparable to the bulk CTL population, which contained 5.8% 1A7-positive CTLs. Taken together, the functional observations from cold target inhibition experiments as well as the receptor coexpression studies provide evidence that the same population of cells is responsible for killing both EBV and tumor targets, and that this effect is associated with coexpression of EBV-specific and tumor-specific receptors on the same cells.

### Example 16

### Chimeric Receptor-Modified CTLs are Rescued to Proliferate by Stimulation with Autologous EBV-LCL

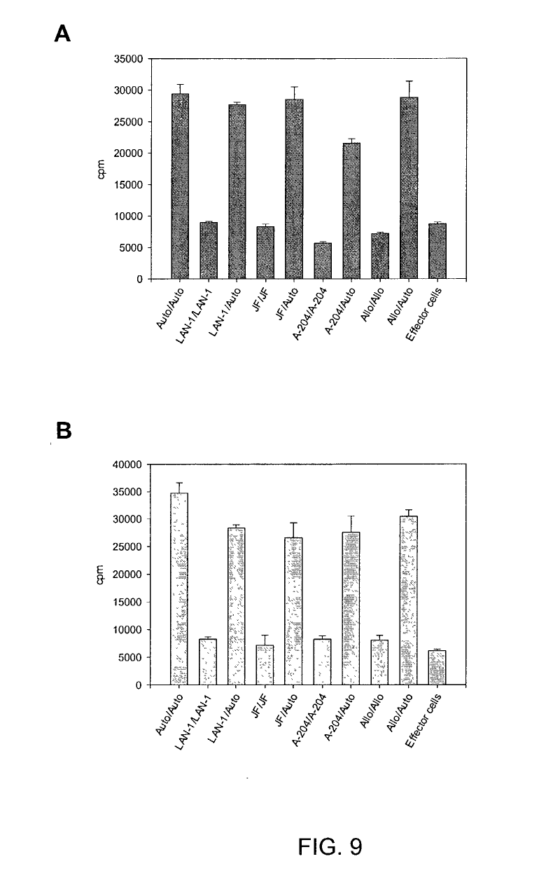

The clinical success of transduced EBV-CTL will likely depend in part upon these cells being able to proliferate when restimulated by autologous EBV targets following exposure to tumor cells. The proliferative responses of the CTLs to tumor targets and to EBV targets were compared after repeated stimulation with either autologous LCL, allogeneic LCL, G_{D2}⁺ tumor cells, or G_{D2}⁻ tumor cells, and in the absence of stimulation. Confirming previous data, autologous LCL alone were capable of inducing the effector cells to proliferate above their low background level.. However, CTL exposed to tumor cells for 1-2 weeks still showed a strong proliferative response when restimulated with autologous LCL, comparable to the response obtained in cells receiving weekly stimulations with EBV targets (FIG. 9).

### Example 17

### Significance of the Present Invention

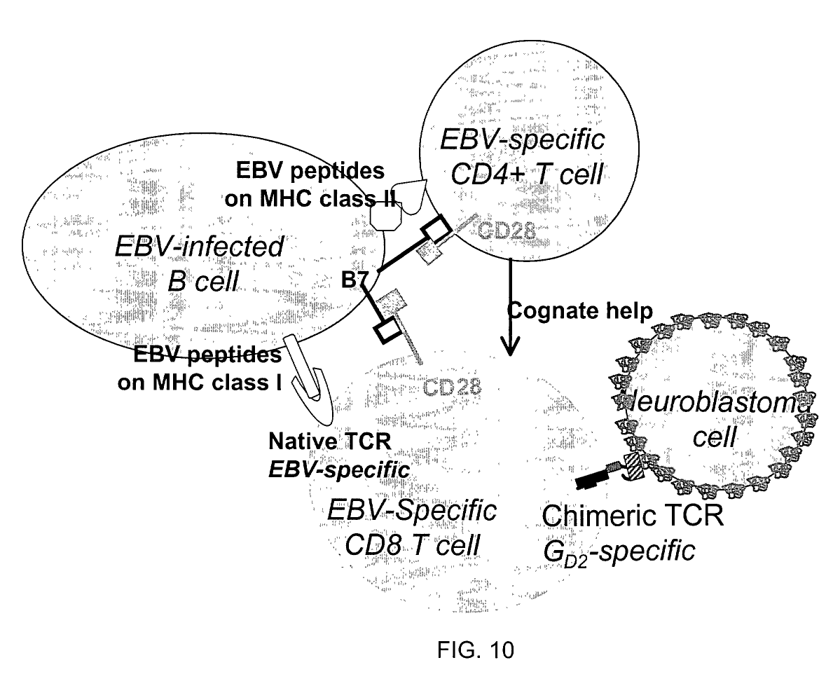

Taken together, the above results indicate that efficient and sustained expansion/activation of transfused chimeric effector T lymphocytes in vivo will require 1) T-cell helper activity provided in a cognate fashion; 2) signaling through the native TCR/CD3 complex and 3) the presence of costimulatory signals and cytokines. The results provided herein indicates that the introduction of chimeric receptors into ex vivo-generated EBV-specific T cell lines will meet all of these requirements (FIG. 10). These cell lines contain antigen-specific CD4⁺ helper T cells that contribute to immune control of EBV latency by providing growth factors capable of maintaining both CD4⁺ and CD8⁺ cells, as well as CD8⁺ cytotoxic T cells (Rooney et al., 1998). The target cells are EBV-positive B lymphocytes, which present antigens extremely well. They express both class I and class II MHC-restricted antigenic epitopes, facilitating cognate interactions between CD4⁺ and CD8⁺ T cells, and are rich in costimulator molecule expression (Heslop et al., 1996; Roskrow et al., 1998; Rooney et al., 1995; Rooney et al., 1998).

What is the evidence that the properties of such T cell lines will be reiterated in vivo? In patients given gene-marked EBV-specific CTLs, a high degree of in vivo expansion is detectable, resulting in long-term persistence and antiviral activity for more than 6 years (Heslop et al., 1996; Roskrow et al., 1998; Rooney et al., 1995; Rooney et al., 1998). Expression of chimeric receptor genes in EBV-specific CTL does not interfere with the cells' ability to proliferate or to respond to autologous EBV-infected targets (FIGS. 3a, 3b). Their ability to kill tumor cell targets through the chimeric receptor is retained even after expansion driven through the EBV-antigen specific native receptors (Table 1, FIG. 5). Following exposure to tumor cells in culture, transduced CTL can be rescued to proliferate and expand by stimulation through their EBV-specific receptor (FIG. 9). In the system described herein, none of the tumor cell targets proved toxic to the CTL or inhibitory to their expansion. Thus, in some embodiments, as a result of the continued presence of viral antigens in an EBV-infected host, engineered antitumor T lymphocytes with native specificity for EBV antigens will survive for extended periods. Such an effect can result only if the same T cell is both EBV- and tumor-specific, a requirement that was met in the present study, both phenotypically (as demonstrated by fluorescent analysis with an anti-idiotype MAb and an EBV tetramer) and functionally (as shown by cross-inhibition of each target cell with either EBV-infected B lymphocytes or tumor cells). While cross inhibition can be demonstrated in short-term assays in vitro, it should not prove a significant limitation in vivo since a single T cell can kill multiple cellular targets sequentially, disengaging from each once killing has been achieved. This effect is illustrated by the ability of chimeric EBV-CTLs that have been repeatedly stimulated by EBV-LCLs, to subsequently kill tumor targets (FIG. 4). Hence, after infusion of chimeric EBV-specific T cells into EBV-positive individuals, there should be lifelong in vivo restimulation via the native TCR in the presence of adequate costimulation as illustrated in FIG. 7. This will prevent functional inactivation of the cells and should enable them to continuously lyse any chimeric receptor target cells they encounter.

Recent evidence suggests that expansion and functional maintenance of these cells will occur even in patients with “normal” levels of EBV DNA and without evident EBV+ malignancy (Wandinger, Neurology 2000; Sarid, J Med Virol 2001; Glaser, Brain Behav Immun 1999). This effect is probably a consequence of periodic reactivation of EBV. Serological evidence suggests that reactivation after primary infection is a frequent event (Wandinger, Neurology 2000; Sarid, J Med Virol 2001; Glaser, Brain Behav Immun 1999). Should the number of functionally activated EBV-specific CTL and their level of activation in the absence of massive EBV reactivation prove to be too low to provide a stimulus of sufficient strength for chimeric-mediated tumor cell lysis, in some embodiments T cell responses are boosted by immunization with autologous irradiated LCL.

A skilled artisan recognizes that the above strategy may be used for any tumor target to which a chimeric receptor can be made. However, one of the major advantages of chimeric T cell-based therapies is that they obviate the need to select and expand the scanty tumor specific T cells present in the circulation. The EBV-specific CTL chimeras described herein would seem to remove that advantage, since an antigen specific selection and expansion process will be required after all. However, a skilled artisan recognizes that the expansion of EBV CTLs and the expansion of tumor specific CTL are two quite separate propositions. The high frequency of EBV-specific precursor cells in peripheral blood and the excellent antigen-presenting capacity of EBV-infected B cells makes this a robust system. Over the past 6 years, there has been successful generation of EBV-specific CTL lines from 138 of 140 donors, including cancer patients pretreated with chemotherapy (Nash et al., 1996). More than 100 patients with EBV-associated infections or malignancy have received EBV-CTL infusions with no serious adverse effects. These results have been confirmed by others (Lucas et al., 2000). Moreover, because the infused cells are expected to expand markedly in vivo and persist in the circulation for extended periods, in some embodiments only limited numbers of cells may need to be grown and infused (Heslop et al., 1996; Roskrow et al., 1998; Rooney et al., 1995; Rooney et al., 1998). The use of EBV-specific cell lines is to be preferred to the use of clones because (1) they are simpler to prepare; (2) the combination of CD4 and CD8 cells present produce a more sustained immune response than CD8⁺ clones alone (Heslop et al., 1996; Rooney et al., 1998; Walter et al., 1995); and (3) EBV antigen escape mutants are less likely to arise (Matloubian et al., 1994). Since high-efficiency cytolysis was achieved with cultures containing up to 90% nontransduced CTLs, there would be no need to coexpress marker or selection genes—a major source of immunogenicity after chimeric T lymphocyte infusion. The individual components of this proposed system have already been safely used in humans; the chimeric receptor in the form of monoclonal antibodies administered to patients with malignancy (Frost et al., 1997; Barker et al., 1991), and the EBV-CTLs given to patients at risk of EBV-lymphoma or with Hodgkin disease (Rooney et al., 1995; Schulz et al., 1984). The findings described herein demonstrate that combining these components as antitumor chimeric receptors expressed by EBV-specific T cells overcomes many of the current limitations of chimeric T-cell immunotherapy.

### Example 18

### Cell Lines and Antibodies

The ecotropic packaging cell line Phoenix (Kinsella et al., 1996) was provided by Gary P. Nolan, Stanford. PG-13, K-562, Raji, Daudi, and Reh cells were obtained from the American Type Culture Collection.

### Example 19

### Construction of Chimeric Receptor Genes

The variable domains of monoclonal antibody FMC-63, specific for CD19, were subcloned as single-chain Fv (scFv) into pRSV-γ² (provided by Z. Eshhar, Rehovot, Isreal), in frame with a sequence encoding the human IgG1 hinge domain and the transmembrane and cytoplasmic domains of the Fc receptor γ chain. The human ζ chain transmembrane and cytoplasmic portions were amplified from pGEM3zζ (Weissman et al., 1988). The chimeric genes were subcloned into the BamHI and NcoI sites of the retroviral vector SFG (Riviere et al., 1995) (provided by R. C. Mulligan, Cambridge, Mass.).

### Example 20

### Quantification of the Transduction Rate by Real-Time Polymerase Chain Reaction

Genomic DNA was isolated from transduced CTL by isopropanol precipitation following cell lysis. For quantification of the transduction rate, a real-time polymerase chain reaction assay was performed accoring to standard methods in the art. PCR amplification was performed with 2× Taqman® Universal Master Mix (PE Applied Biosystems), and using the ABI PRISM 7700® Sequence Detection System (PE Applied Biosystems).

### Example 21

### CD19ζ-Transduced EBV-Specific CTLs Express the Chimeric Receptor while Maintaining Their Immunophenotype

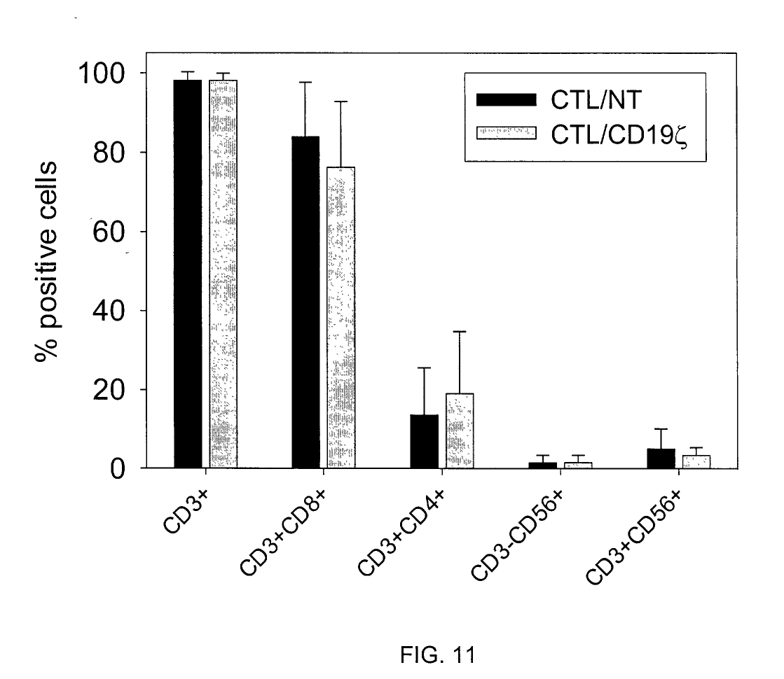

Seven EBV-specific CTL lines, generated from four different seropositive healthy donors (Rooney et al., 1995; Rooney et al., 1998), were transduced with SFG/CD19ζ chimeric receptor genes. This receptor is derived from the FMC-63 monoclonal antibody, which recognizes CD19, a B lymphocyte cell surface marker. Transduction efficiency was monitored by methods described in Example 20. Transduction efficiencies of CTL transduced with SFG/CD19ζ were 23-67% (mean 36%). Expression of chrec RNA in transduced CT llines was confirmed by reverse transcriptase PCr analysis. The CTL linesgenerated had a characteristic phenotype with 98-100% CD3⁺ T cells, of which 62-99% coexpressed CD8 (mean of 84%), whereas 1-38% (mean of 14%) had a T helper cell phenotype (CD3⁺CD4⁺). Following transduction, no major changes of cellular immunophenotypes were observed by comparison with nontransduced cells (FIG. 11).

### Example 22

### CD19ζ-Expressing CTL Efficiently and Specifically Lyse Both EBV-LCL and CD19⁺ Tumor Targets

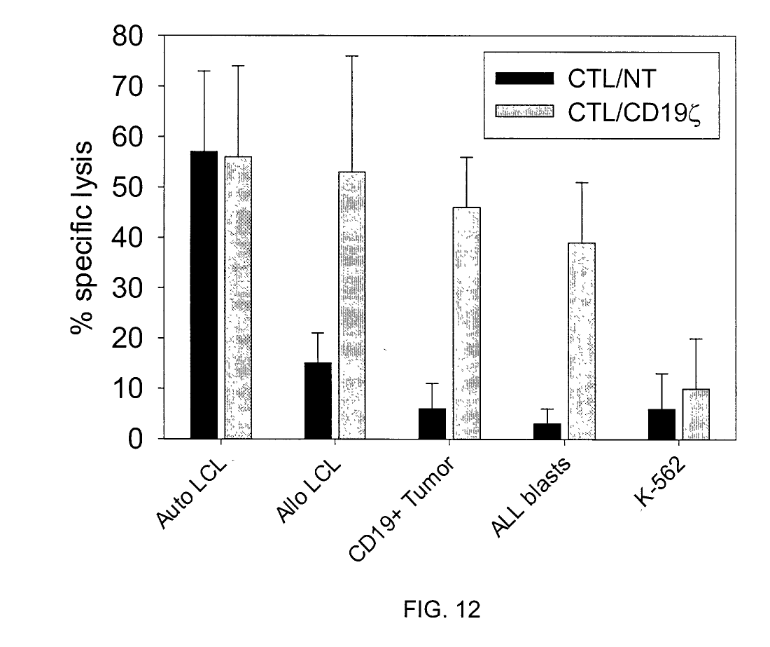

The cytotoxic activity of CD19ζ-transduced CTL and nontransduced CTL was compared in standard ⁵¹Cr release assays. CD19ζ-transduced CTL maintained their cytolytic activity against autologous EBV targets, with a mean specific lysis of autologous EBV-LCL of 57±16% by nontransduced cells compared to 56±18% lysis by CD19ζ-transduced CTL (FIG. 12). The ability of CD19ζ-transduced CTL to lyse CD19⁺ tumor targets was tested in the Burkitt's lymphoma cell line Raji, the CD19⁺ acute lymphoblastic leukemia line Reh, and against CD19⁺ primary leukemic blast cells from a pediatric patient. None of the nontransduced CTL lines had significant reactivity with any of the tumor targets. In Raji and Reh cells, 37-66% (mean 46%) were specifically lysed by CD19ζ-transduced CTL. In the leukemic blast cells, the percentage of cells lysed by CD19ζ-transduced CTL was 30-47% (mean 39%). CD19ζ-transduced CTL had no significant cytotoxic activity against K-562, a CD19-negative erytholeukemia cell line (FIG. 12).

### Example 23

### CD19ζ-Mediated Tumor Cell Recognition is Mediated by Surface CD19 and is Non-MHC-Restricted.

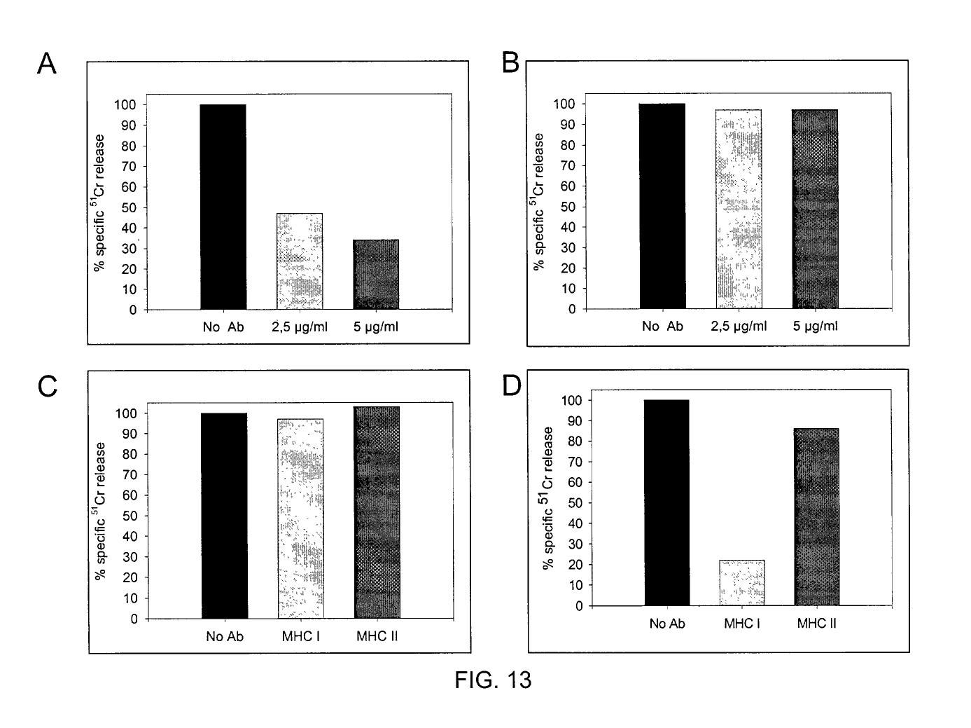

Preincubation of CD19⁺ tumor target cells with CD19-specific monoclonal antibody FMC-63 resulted in up to 66% inhibition of lysis by CD19ζ-transduced CTL, indicating a CD19-mediated mechanism of recognition (FIG. 13A). Lysis of autologuos LCL by nontransduced CTL was not affected by blocking of surface CD19 (FIG. 13B). Inhibition studies were performed using antibodies against monomorphic determinants of HLA class I and II to exclude an MHC-dependent mechanism of lysis of tumor target cells. MHC class I was identified as the major restriction element for autologous LCL lysis by nontransduced CTL lines (FIG. 13D). Precincubation with HLA class I and II blocking antibodies did not affect the lysis of Raji cells by transduced CTL, indicating a lack of MHC restriction (FIG. 13C).

### Example 24

### CD19ζ-Expressing CTL Recognize Mismatched LCL Via the Chimeric Receptor and Autologous LCL Via Both Their Native T Cell Receptor and the Chimeric Receptor.

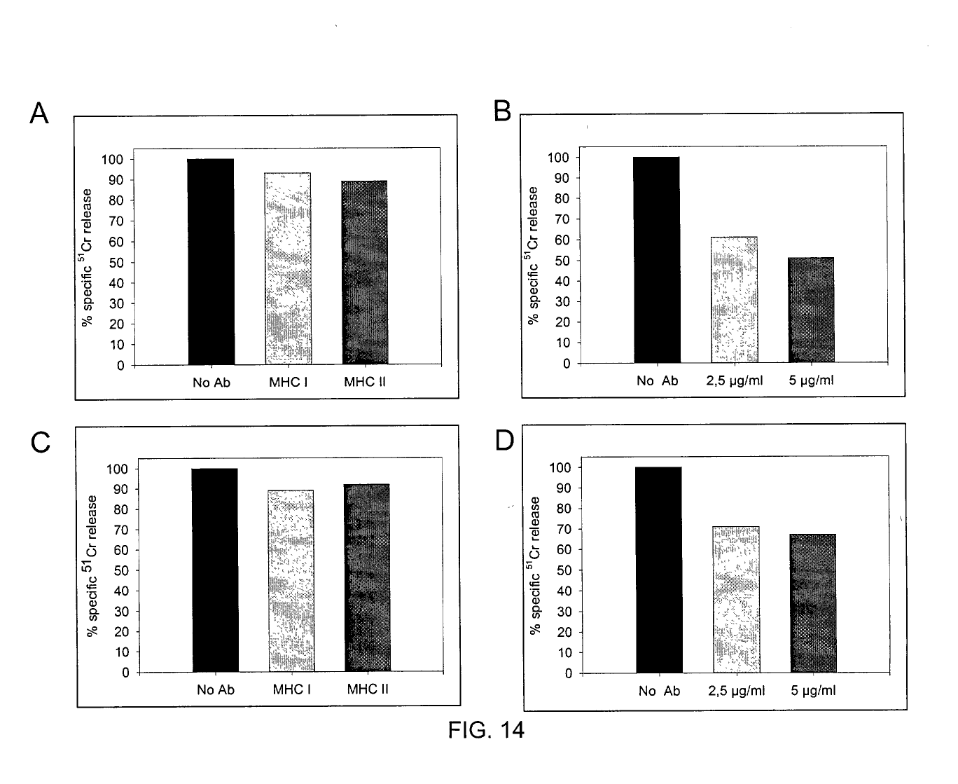

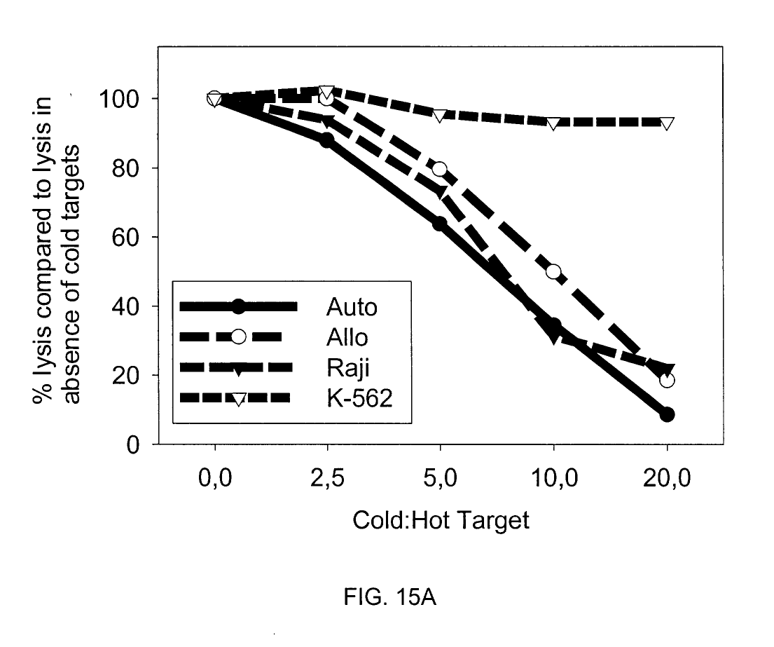

In further antibody blocking experiments, the lysis of HLA-mismatched allogeneic LCL by CD19ζ-transduced CTL by chRec was tested. Lysis of mismatched LCL was not significantly inhibited by HLA class I and II antibodies, but up to 49% inhibition of lysis was obtained by preinvubation with anti-CD19 mAb. (FIG. 14A and 14B). This confirmed a chRec, non-MHC-restricted mechanism of recognition and cytolysis comparable to the one observed with CD19⁺ tumor targets. Preincubation of CD19ζ-transduced CTL with MHC class I mAb resulted in no significant inhibition of autologous LCL lysis (FIG. 14C). However, up to 33% of autologous LCL lysis was blocked by preincubation with an anti-CD19 mAb (FIG. 14D). Thus, the chimeric receptor appears to contribute significantly to autologous EBV-LCL lysis by CD19ζ-expressing CTL, and blocking of either native or chRec-mediated lysis can at least partly be compensated for by the alternative receptor.

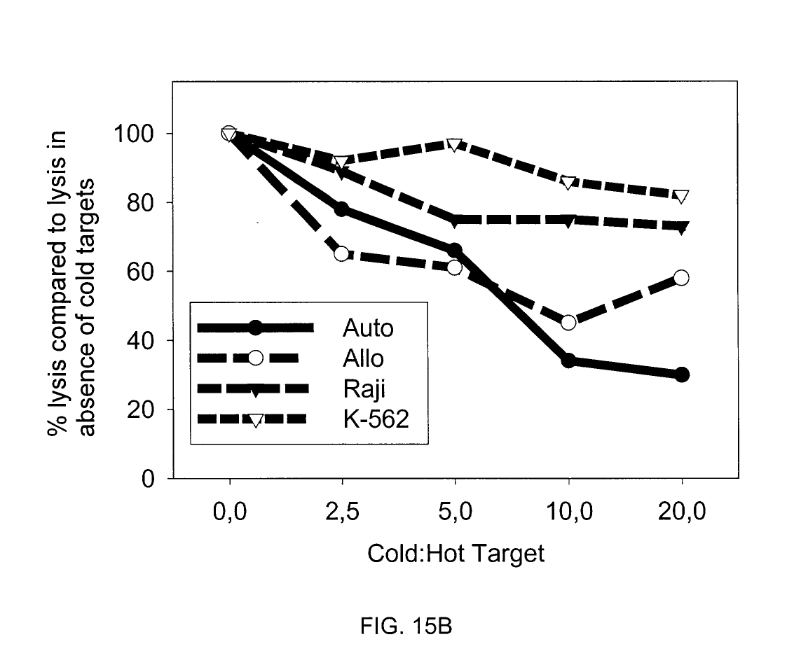

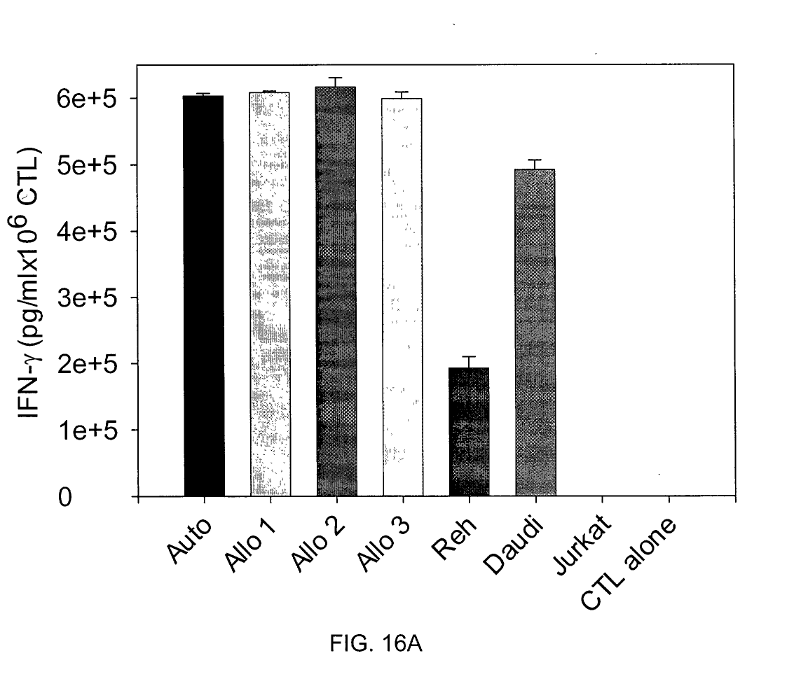

Cold target inhibition assays were performed to elucidate the contributory roles of both the native and the chimeric T cell receptor to the cytotoxic activity of the transduced CTL. The antigen specificity of target cell lysis by gene modified EBV-specific CTL was determined by analyzing the capacity of unlabeled CD19⁺ tumor cells and LCL to block lysis of autologous and allogeneic LCL respectively. Addition of unlabeled Raji cells or autologous LCL significantly (p<0.05) inhibited the lysis of allogeneic EBV targets by CD19ζ CTL when compared to maximum inhibition obtained by adding unlabeled allogeneic LCL (FIG. 15A). In contrast, the cytotoxic activity of the gene-modified CTL towards autologous LCL was only incompletely inhibited by competition from nonlabeled tumor cells and allogeneic LCL (FIG. 15B). CD19-negative cells had no effect on the lysis of autologous and allogeneic LCL (FIGS. 15A and 15B). Functional observations from inhibition studies with unlabeled target cells and with monoclonal antibodies both suggest that allogeneic LCL and tumor cell killing by the gene-modified cells is mediated by the chimeric receptor alone, whereas autologous EBV targets are recognized via peptide presented on MHC, as well as via surface CD19, with both the native receptor and the chimeric receptor being coexpressed on the same cells.

### Example 25

### CD19ζ CTL Fail to Proliferate in Response to CD19⁺ Tumor Target Cells while Responding to Allogeneic LCL Stimulation

D19ζ/EBV-dual-specific CTL was used as a model system for comparing the effect of the cellular context of target antigen expression on the function of the chimeric T cell receptor. Whereas most tumor cells lack adequate costimulation to induce a complete T cell activation response, lymphoblastoid B cells are excellent APC that express a wide variety of costimulatory molecules.

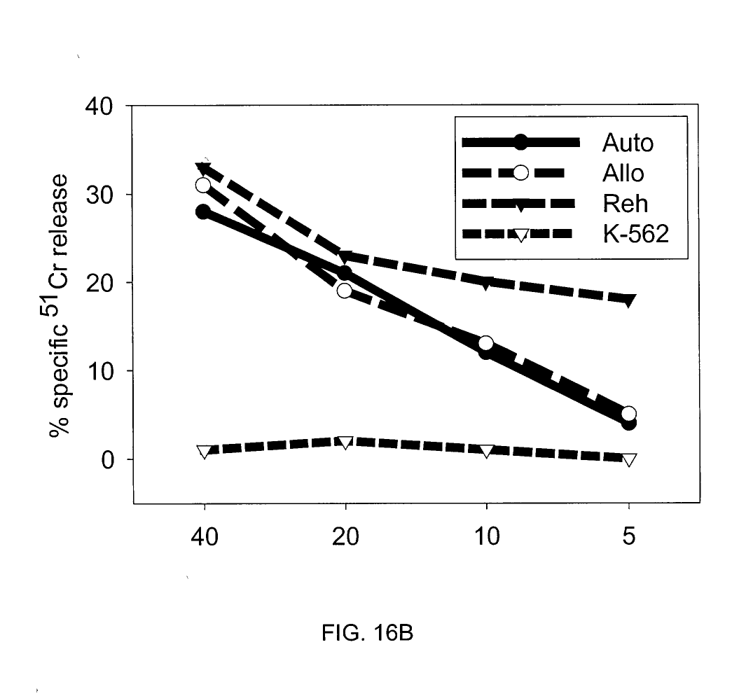

To obtain a pure population of CTL expressing the chRec transgene, CTL clones were obtained by single-cell cloning of bulk transduced cells and subsequent expansion in the presence of irradiated autologous LCL and allogeneic mononuclear cells. CD19ζ⁺ and CD19ζ⁻ clones were identified by screening for IFN-γ production in response to stimulation with CD19⁺ tumor cells. All clones had similar phenotypes with 100% CD4⁺CD3⁺CD56⁻ CTL. FIG. 16A shows that whereas clone #11 responded to coincubation with CD19ζ⁺ tumor and CD19⁺, HLA-mismatched EBV targets with secretion of IFN-γ at quantities comparable to those obtained by coculture with autologous EBV-LCL), IFN-γ production by clone #10 was restricted to stimulation with autologous LCL, suggesting absence of the chimeric receptor transgene. The presence of the CD19ζ transgene in CTL clone #11 was confirmed by quantitative PCR, demonstrating 100% transduction efficiency. In contrast, CD19ζ could not be detected in genomic DNA of clone #10. As expected, clone 11 showed specific lysis of CD19⁺ targets as well as autologous EBV-LCL (FIG. 16B).

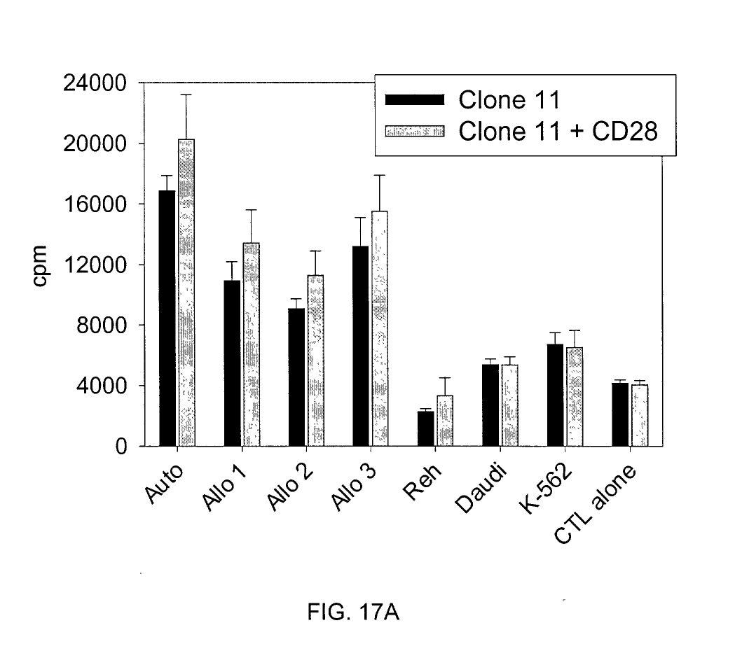

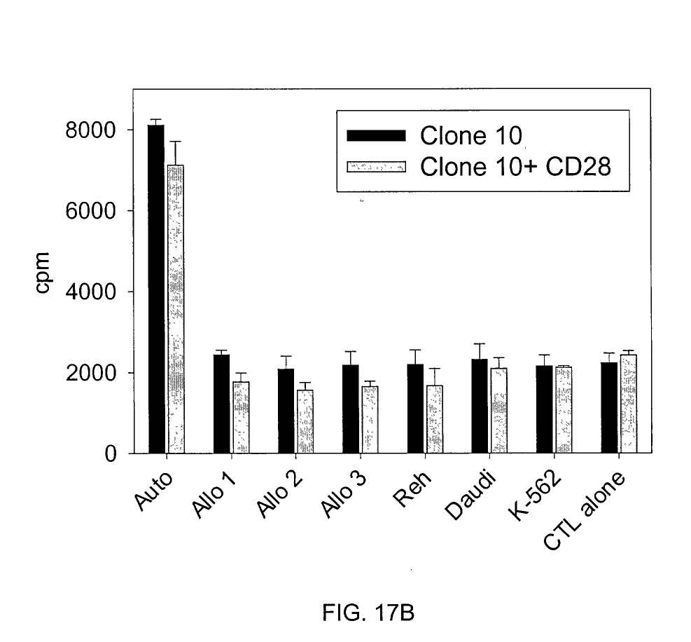

To induce target-specific T cell proliferation, CD19ζ+ (clone#11) and CD19ζ− (clone #10) CTL as well as nontransduced bulk CTL from the same donor were cultured in the presence of irradiated autologous or allogeneic LCL, CD 19+ (Reh, Daudi) and D19− (K-562) tumor cell targets and rhIL-2 (50 IU/ml). Whereas coculture with autologous LCL triggered substantial [³H] thymidine uptake by both CTL clones, incubation with the tumor targets did not elicit a proliferative response above background in either clone (FIG. 17) nor in nontransduced bulk CTL. However, coculture of clone #11 with LCL lines obtained from three HLA mismatched allogeneic donors induced substantial [³H] thymidine uptake at 54-80% of that observed with autologous LCL. The CD19-negative clone, in contrast, failed to proliferate specifically in response to allogeneic LCL.

The best-characterized costimulatory signal is the one delivered by ligation of the CD28 receptor on T cells. To investigate the effect of CD28 on the ability of chimeric receptors to induce T cell proliferation, tritiated thymidine uptake in response to CD19-positive target cells was compared in the presence and absence of immobilized anti-CD28 antibody (1 μg/ml). CD28 crosslinking had no effect on the proliferative CTL response to either target (FIG. 17). In particular, the failure of tumor cells to induce CTL proliferation persisted in the presence of immobilized anti-CD28 antibody.

Thus, the inability of the chimeric receptor to induce specific CTL proliferation can largely be overcome by expressing the target antigen on a professional APC, but is unaffected by crosslinking of CD28 alone.

### Example 26

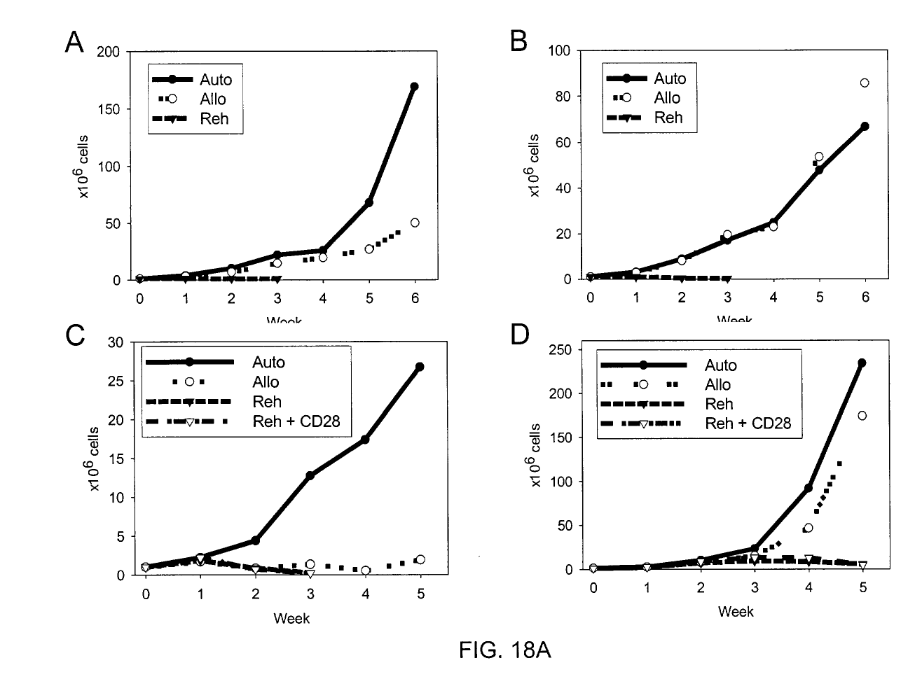

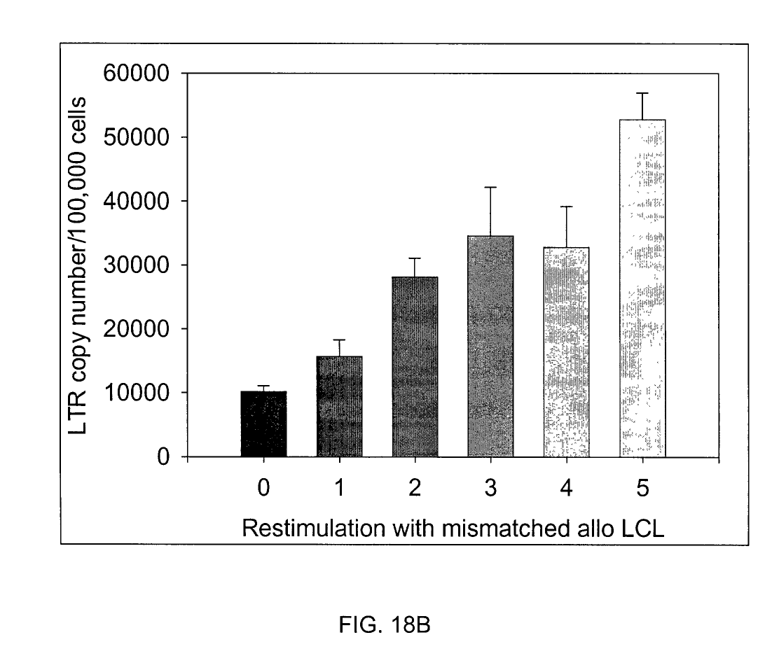

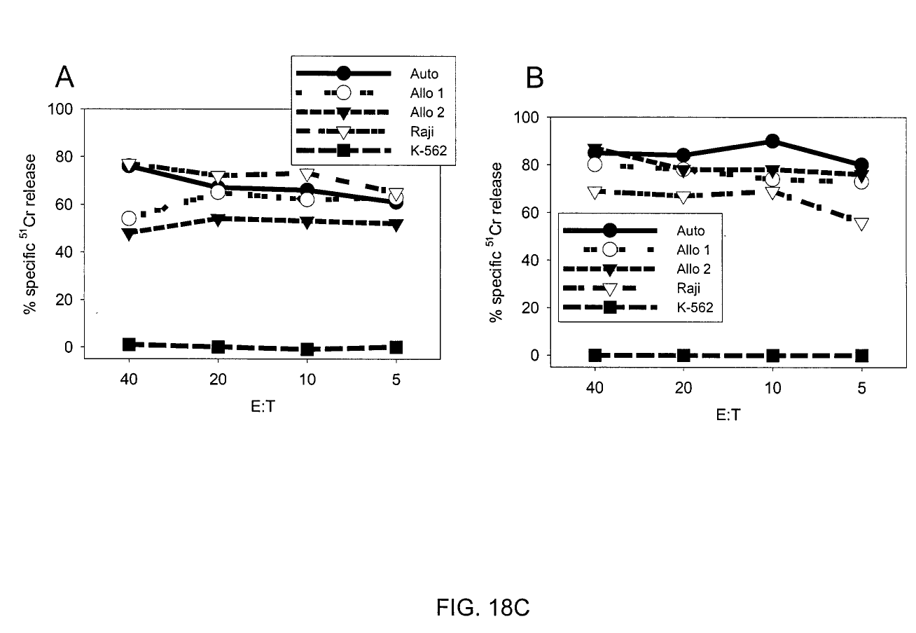

### CD19ζ-Transduced Bulk CTL Expand In Response to Autologous and Allogeneic LCL but Not to CD19⁺ Tumor Targets

Whereas transduced bulk CTL continued to expand in response to stimulation with irradiated autologous LCL, with kinetics similar to those of nontransduced cells, neither the nontransduced nor the transduced CTL could be maintained in culture for longer than 3 weeks when stimulated with CD19⁺ tumor cells (FIG. 18A). Furthermore, CD28 crosslinking using immobilized CD28-specific monoclonal antibody did not result in prolonged survival of the CTL. In contrast, coculture with mismatched allogeneic LCL promoted expansion of CD19ζ-expressing CTL similar to that observed with autologous LCL, significantly exceeding non-specific background expansion observed when nontransduced CTL were maintained in the presence of allogeneic LCL.

To further demonstrate the selective enrichment of CD19ζ-expressing CTL by LCL stimulation of the chimeric receptor, weekly PCR quantifications of transgene copy number in bulk CTL during expansion were performed. A consistent increase of transgene copy number in CTL maintained in the presence of mismatched allogeneic LCL was observed (FIG. 18B). CD19ζ-expressing CTL expanded by chimeric receptor stimulation maintained efficient cytolysis of autologous EBV targets as well as CD19⁺ tumor cells and mismatched LCL that was comparable to lysis by CTL restimulated through their native receptor (FIG. 18C). Thus, engagement of the chimeric receptor by CD19 expressed in the context of a professional APC appears to confer a selective growth advantage, resulting in overgrowth of the bulk population of transduced cells by chRec-positive CTL that maintain their native receptor specificity.

### REFERENCES

All patents and publications mentioned in the specification are indicative of the levels of those skilled in the art to which the invention pertains. All patents and publications are herein incorporated by reference to the same extent as if each individual publication was specifically and individually indicated to be incorporated by reference.

PATENTS

| | Issued |
|---|---|
| U.S. Pat. No., 5,359,046 | Oct. 25, 1994 |
| U.S. Pat. No., 5,827,642 | Oct. 27, 1998 |
| WO 00/31239 | Jun. 2, 2000 |
| WO 93/19163 | Sep. 30, 1993 |

### PUBLICATIONS

Abken H, Hombach A, Heuser C, Reinhold U. A novel strategy in the elimination of disseminated melanoma cells: chimeric receptors endow T cells with tumor specificity. Recent Results Cancer Res. 2001; 158:249-64.

Altenschmidt U, Kahl R, Moritz D, Schnierle B S, Gerstmayer B, Wels W, Groner B. Cytolysis of tumor cells expressing the Neu/erbB-2, erbB-3, and erbB-4 receptors by genetically targeted naive T lymphocytes. Clin Cancer Res. June 1996; 2(6):1001-8.

Altenschmidt U, Klundt E, Groner B. Adoptive transfer of in vitro-targeted, activated T lymphocytes results in total tumor regression. J Immunol. 1997;159:5509-5515.

Annenkov A E, Moyes S P, Eshhar Z, Mageed R A, Chemajovsky Y. Loss of original antigenic specificity in T cell hybridomas transduced with a chimeric receptor containing single-chain Fv of an anti-collagen antibody and Fc epsilonRI-signaling gamma subunit. J Immunol. December 1998 15;161(12):6604-13.

Ausubel, et al., 1987.

Barker E, Mueller B M, Handgretinger R, Herter M, Yu A L, Reisfeld R A. Effect of a chimeric anti-ganglioside GD2 antibody on cell-mediated lysis of human neuroblastoma cells. Cancer Res. 1991;51:144-149.

Beaucage and Carruthers (1981) Tetra. Letts. 22:1859-1862.

Bitton N, Verrier F, Debre P, Gorochov G. Characterization of T cell-expressed chimeric receptors with antibody-type specificity for the CD4 binding site of HIV-1 gp120. Eur J Immunol. December 1998; 28(12):4177-87.

Brocker T, Karjalainen K. Signals through T cell receptor-zeta chain alone are insufficient to prime resting T lymphocytes. J Exp Med. 1995;181:1653-1659.

Brocker T. Chimeric Fv-zeta or Fv-epsilon receptors are not sufficient to induce activation or cytokine production in peripheral T cells. Blood. 2000;96:1999-2001.

Brodie S J, Lewinsohn D A, Patterson B K, et al. In vivo migration and function of transferred HIV-1-specific cytotoxic T cells. Nat Med. 1999;5:34-41.

Cann et al., Oncogene 3:123, 1988

Cardin R D, Brooks J W, Sarawar S R, Doherty P C. Progressive loss of CD8+ T cell-mediated control of a gamma-herpesvirus in the absence of CD4+ T cells. J Exp Med. 1996;184:863-871.

Carter, B J, Current Opinion in Biotechnology, 1992, 3:533-539.

Darcy P K, Kershaw M H, Trapani J A, Smyth M J. Expression in cytotoxic T lymphocytes of a single-chain anti-carcinoembryonic antigen antibody. Redirected Fas ligand-mediated lysis of colon carcinoma. Eur J Immunol. May 1998; 28(5):1663-72.

Deans et al., 1984.

Eshhar Z, Waks T, Gross G, Schindler D G. Specific activation and targeting of cytotoxic lymphocytes through chimeric single chains consisting of antibody-binding domains and the gamma or zeta subunits of the immunoglobulin and T-cell receptors. Proc Natl Acad Sci U S A. 1993;90:720-724.

Evavold B D, Allen P M. Separation of IL-4 production from Th cell proliferation by an altered T cell receptor ligand. Science. 1991;252:1308-1310.

Frost J D, Hank J A, Reaman G H, et al. A phase I/IB trial of murine monoclonal anti-G_{D}2 antibody 14.G2a plus interleukin-2 in children with refractory neuroblastoma: a report of the Children's Cancer Group. Cancer. 1997;80:317-333.

Glaser R, Friedman S B, Smyth J, et al. The differential impact of training stress and final examination stress on herpesvirus latency at the United States Military Academy at West Point. Brain Behav Immun. 1999; 13:240-251.

Goding, 1986, pp. 65-66, 71-74.

Gottschalk S, Ng C Y, Perez M, et al. An Epstein-Barr virus deletion mutant associated with fatal lymphoproliferative disease unresponsive to therapy with virus-specific CTLs. Blood. 2001;97:835-843.

Gustafsson A, Levitsky V, Zou J Z, et al. Epstein-Barr virus (EBV) load in bone marrow transplant recipients at risk to develop posttransplant lymphoproliferative disease: prophylactic infusion of EBV-specific cytotoxic T cells. Blood. 2000;95:807-814.

Hege K M, Cooke K S, Finer M H, Zsebo K M, Roberts M R. Systemic T cell-independent tumor immunity after transplantation of universal receptor-modified bone marrow into SCID mice. J Exp Med. Dec. 1, 1996; 184(6):2261-9.

Hemmer B, Vergelli M, Tranquill L, et al. Human T-cell response to myelin basic protein peptide (83-99): extensive heterogeneity in antigen recognition, function, and phenotype. Neurology. 1997;49:1116-1126.

Heslop H E, Ng C Y, Li C, et al. Long-term restoration of immunity against Epstein-Barr virus infection by adoptive transfer of gene-modified virus-specific T lymphocytes. Nat Med. 1996;2:551-555.

Hombach A, Heuser C, Sircar R, Tillmann T, Diehl V, Kruis W, Pohl C, Abken H. T cell targeting of TAG72+ tumor cells by a chimeric receptor with antibody-like specificity for a carbohydrate epitope. Gastroenterology. October 1997; 113(4): 1163-70.

Hombach A, Heuser C, Sircar R, Tillmann T, Diehl V, Pohl C, Abken H. An anti-CD30 chimeric receptor that mediates CD3-zeta-independent T-cell activation against Hodgkin's lymphoma cells in the presence of soluble CD30. Cancer Res. Mar. 15, 1998; 58(6):1116-9.

Hombach A, Heuser C, Sircar R, Tillmann T, Diehl V, Pohl C, Abken H. Characterization of a chimeric T-cell receptor with specificity for the Hodgkin's lymphoma-associated CD30 antigen. J Immunother. November 1999; 22(6):473-80.

Hwu P, Yang J C, Cowherd R, et al. In vivo antitumor activity of T cells redirected with chimeric antibody/T-cell receptor genes. Cancer Res. 1995;55:3369-3373.

Karlsson et al., Proc. Natl. Acad. Sci. USA 84 82:158, 1985.

Karlsson et al., EMBO J. 5:2377, 1986.

Kasid A, Morecki S, Aebersold P, Cometta K, Culver K, Freeman S, Director E, Lotze M T, Blaese R M, Anderson W F, et al. Human gene transfer: characterization of human tumor-infiltrating lymphocytes as vehicles for retroviral-mediated gene transfer in man. Proc Natl Acad Sci U S A. January 1990; 87(1):473-7.

Khanna R, Burrows S R. Role of cytotoxic t lymphocytes in epstein-barr virus-associated diseases [In Process Citation]. Annu Rev Microbiol. 2000;54:19-48.

Kinsella T M, Nolan G P. Episomal vectors rapidly and stably produce high-titer recombinant retrovirus. Hum Gene Ther. 1996;7:1405-1413.

Kohler G, Milstein C. Continuous cultures of fused cells secreting antibody of predefined specificity. Nature. Aug. 7, 1975; 256(5517):495-7.

Kohler G, Milstein C. Derivation of specific antibody-producing tissue culture and tumor lines by cell fusion. Eur J Immunol. July 1976; 6(7):511-9.

Krause A, Guo H F, Latouche J B, Tan C, Cheung N K, Sadelain M. Antigen-dependent CD28 signaling selectively enhances survival and proliferation in genetically modified activated human primary T lymphocytes. J Exp Med. 1998;188: 619-626.

LaFace D, Hermonat P, Wakeland E, Peck A. Gene transfer into hematopoietic progenitor cells mediated by an adeno-associated virus vector. Virology. February 1988; 162(2):483-6.

Matteucci et al. (1981) J. Am. Chem. Soc. 103:3185

Matloubian M, Concepcion R J, Ahmed R. CD4+ T cells are required to sustain CD8+ cytotoxic T-cell responses during chronic viral infection. J Virol. 1994;68:8056-8063.

McGuinness R P, Ge Y, Patel S D, Kashmiri S V, Lee H S, Hand P H, Schlom J, Finer M H, McArthur J G. Anti-tumor activity of human T cells expressing the CC49-zeta chimeric immune receptor. Hum Gene Ther. Jan. 20, 1999; 10(2):165-73.

Mitsuyasu R T, Anton P A, Deeks S G, et al. Prolonged survival and tissue trafficking following adoptive transfer of CD4zeta gene-modified autologous CD4(+) and CD8(+) T cells in human immunodeficiency virus-infected subjects. Blood. 2000;96:785-793.

Moritz D, Groner B. A spacer region between the single chain antibody- and the CD3 zeta-chain domain of chimeric T cell receptor components is required for efficient ligand binding and signaling activity. Gene Ther. October 1995; 2(8):539-46.

Moritz D, Wels W, Mattem J, Groner B. Cytotoxic T lymphocytes with a grafted recognition specificity for ERBB2-expressing tumor cells. Proc Natl Acad Sci U S A. May 10, 1994; 91(10):4318-22.

Mujoo K, Kipps T J, Yang H M, et al. Functional properties and effect on growth suppression of human neuroblastoma tumors by isotype switch variants of monoclonal antiganglioside G_{D}2 antibody 14.18. Cancer Res. 1989;49:2857-2861.

Nash R A, Pineiro L A, Storb R, et al. FK506 in combination with methotrexate for the prevention of graft-versus-host disease after marrow transplantation from matched unrelated donors. Blood. 1996;88:3634-3641.

Nishihara et al., Cancer Res. 48:4730, 1988.

Overell et al., Oncogene 4:1425, 1989.

Ren-Heidenreich L, Hayman G T, Trevor K T. Specific targeting of EGP-2+ tumor cells by primary lymphocytes modified with chimeric T cell receptors. Hum Gene Ther. Jan. 1, 2000; 11(1):9-19.

Reuther et al., Mol. Cell Biol. 6:123, 1986.

Riddell, S., et al., Human Gene Therapy 3:319-338, 1992.

Riviere I, Brose K, Mulligan R C. Effects of retroviral vector design on expression of human adenosine deaminase in murine bone marrow transplant recipients engrafted with genetically modified cells. Proc Natl Acad Sci U S A. 1995;92:6733-6737.

Roberts M R, Cooke K S, Tran A C, Smith K A, Lin W Y, Wang M, Dull T J, Farson D, Zsebo K M, Finer M H. Antigen-specific cytolysis by neutrophils and NK cells expressing chimeric immune receptors bearing zeta or gamma signaling domains. J Immunol. 1998 Jul 1;161(1):375-84.

Roberts M R, Qin L, Zhang D, Smith D H, Tran A C, Dull T J, Groopman J E, Capon D J, Byrn R A, Finer M H. Targeting of human immunodeficiency virus-infected cells by CD8⁺ T lymphocytes armed with universal T-cell receptors. Blood. Nov. 1, 1994; 84(9):2878-89.

Romeo C, Seed B. Cellular immunity to HIV activated by CD4 fused to T cell or Fc receptor polypeptides. Cell. Mar. 8, 1991; 64(5):1037-46.

Rooney C M, Smith C A, Ng C Y, et al. Infusion of cytotoxic T cells for the prevention and treatment of Epstein-Barr virus-induced lymphoma in allogeneic transplant recipients. Blood. 1998;92:1549-1555.

Rooney C M, Smith C A, Ng C Y, et al. Use of gene-modified virus-specific T lymphocytes to control Epstein- Barr-virus-related lymphoproliferation. Lancet. 1995;345:9-13.

Rosenberg S A. Immunotherapy of patients with advanced cancer using interleukin-2 alone or in combination with lymphokine activated killer cells. Important Adv Oncol. 1988;:217-57.

Roskrow M A, Suzuki N, Gan Y, et al. Epstein-Barr virus (EBV)-specific cytotoxic T lymphocytes for the treatment of patients with EBV-positive relapsed Hodgkin's disease. Blood. 1998;91:2925-2934.

Sambrook et al., Molecular cloning: a laboratory manual, 2nd Ed., 1989.

Sarid O, Anson O, Yaari A, Margalith M. Epstein-Barr virus specific salivary antibodies as related to stress caused by examinations. J Med Virol. 2001;64:149-156.

Schulz G, Cheresh D A, Varki N M, Yu A, Staffileno L K, Reisfeld R A. Detection of ganglioside GD2 in tumor tissues and sera of neuroblastoma patients. Cancer Res. 1984;44:5914-5920.

Sen G, Chakraborty M, Foon K A, Reisfeld R A, Bhattacharya-Chatterjee M. Preclinical evaluation in nonhuman primates of murine monoclonal anti-idiotype antibody that mimics the disialoganglioside GD2. Clin Cancer Res. 1997;3:1969-1976.

Smith G P. Filamentous fusion phage: novel expression vectors that display cloned antigens on the virion surface. Science. 1985;228:1315-1317.

Stancovski I, Schindler D G, Waks T, Yarden Y, Sela M, Eshhar Z. Targeting of T lymphocytes to Neu/HER2-expressing cells using chimeric single chain Fv receptors. J Immunol. Dec. 1, 1993; 151(11):6577-82.

Tran A C, Zhang D, Byrn R, Roberts M R. Chimeric zeta-receptors direct human natural killer (NK) effector function to permit killing of NK-resistant tumor cells and HIV-infected T lymphocytes. J Immunol. Jul. 15, 1995; 155(2):1000-9.

Walker R E, Bechtel C M, Natarajan V, et al. Long-term in vivo survival of receptor-modified syngeneic T cells in patients with human immunodeficiency virus infection. Blood. 2000;96:467-474.

Walter E A, Greenberg P D, Gilbert M J, et al. Reconstitution of cellular immunity against cytomegalovirus in recipients of allogeneic bone marrow by transfer of T-cell clones from the donor. N Engl J Med. 1995;333:1038-1044.

Wandinger K, Jabs W, Siekhaus A, et al. Association between clinical disease activity and Epstein-Barr virus reactivation in MS. Neurology. 2000;55:178-184.

Weijtens M E, Hart E H, Bolhuis R L. Functional balance between T cell chimeric receptor density and tumor associated antigen density: CTL mediated cytolysis and lymphokine production. Gene Ther. January 2000; 7(1):35-42.

Weijtens M E, Willemsen R A, Valerio D, Stam K, Bolhuis R L. Single chain Ig/gamma gene-redirected human T lymphocytes produce cytokines, specifically lyse tumor cells, and recycle lytic capacity. J Immunol. Jul. 15, 1996; 157(2):836-43.

Weijtens M E, Willemsen R A, van Krimpen B A, Bolhuis R L. Chimeric scFv/gamma receptor-mediated T-cell lysis of tumor cells is coregulated by adhesion and accessory molecules. Int J Cancer. Jul. 17, 1998; 77(2):181-7.

Weiss et al. (Coffin, 1985, pp. 17-71) (eds.), RNA Tumor Viruses, 2nd ed., Vol. 2, Cold Spring Harbor Laboratory, New York.

Weissman A M, Hou D, Orloff D G, et al. Molecular cloning and chromosomal localization of the human T-cell receptor zeta chain: distinction from the molecular CD3 complex. Proc Natl Acad Sci U S A. 1988;85:9709-9713.

Yang O O, Tran A C, Kalams S A, Johnson R P, Roberts M R, Walker B D. Lysis of HIV-1-infected cells and inhibition of viral replication by universal receptor T cells. Proc Natl Acad Sci U S A. Oct. 14, 1997; 94(21):11478-83.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A T lymphocyte, comprising:
an antigen-specific receptor, wherein the presence of said antigen-specific receptor leads to increased in vivo survival of said lymphocyte; and
a chimeric receptor.

2. The lymphocyte of claim 1, wherein the antigen for said antigen-specific receptor comprises a viral polypeptide.

3. The lymphocyte of claim 2, wherein said viral polypeptide is an Epstein Barr Virus polypeptide.

4. The lymphocyte of claim 1, wherein said chimeric receptor further comprises an antigen-binding moiety.

5. The lymphocyte of claim 4, wherein said antigen-binding moiety is a single chain antibody.

6. The lymphocyte of claim 1, wherein said chimeric receptor is an antitumor chimeric receptor.

7. The lymphocyte of claim 1, wherein said antitumor chimeric receptor is 14.G2a-ζ.

8. The lymphocyte of claim 6, wherein said antitumor chimeric receptor is CD19 specific.

9. A cytotoxic T lymphocyte, comprising:
an Epstein Barr Virus-specific receptor, wherein the presence of said receptor leads to increased in vivo survival of said lymphocyte; and
a 14.G2a-ζ chimeric receptor.

10. A cytotoxic T lymphocyte, comprising:
an Epstein Barr Virus-specific receptor, wherein the presence of said receptor leads to increased in vivo survival of said lymphocyte; and
a CD 19 specific chimeric receptor.

11. A population of cytotoxic T lymphocytes, comprising at least one cytotoxic T lymphocyte having:
an antigen-specific receptor, wherein the presence of said antigen-specific receptor leads to increased in vivo survival of said lymphocyte; and
a chimeric receptor.

12. The population of lymphocytes of claim 11, wherein the population comprises CD4⁺ T lymphocytes, CD8⁺ T lymphocytes, or a combination thereof.

13. The population of claim 11, wherein the antigen for said antigen-specific receptor comprises a viral polypeptide.

14. The population of claim 11, wherein said viral polypeptide is an Epstein Barr Virus polypeptide.

15. The population of claim 11, wherein said chimeric receptor is an antitumor chimeric receptor.

16. The population of claim 15, wherein said antitumor chimeric receptor is 14.G2a-ζ.

17. The population of claim 15, wherein said antitumor chimeric receptor is CD19 specific.

18. A method of enhancing activity of a chimeric T lymphocyte in an individual, comprising:
obtaining a T lymphocyte, wherein said T lymphocyte comprises
an antigen-specific receptor, wherein the presence of said antigen-specific receptor leads to increased in vivo survival of said lymphocyte; and
a chimeric receptor; and
administering said cell to said individual.

19. The method of claim 18, wherein said antigen is an Epstein-Barr Virus polypeptide.

20. A method of treating a disease in an individual, wherein said disease is associated with a pathogen or cell having a first antigen, comprising:
obtaining a cytotoxic T lymphocyte, wherein said lymphocyte comprises:
a receptor specific for a second antigen, wherein the presence of said second antigen-specific receptor leads to increased in vivo survival of said lymphocyte; and
a chimeric receptor specific for said first antigen; and
administering said T lymphocyte to said individual.

21. The method of claim 20, wherein said disease is cancer and said first antigen is a tumor-specific or tumor-associated antigen.

22. A method of treating a tumor in an individual, comprising:
obtaining a cytotoxic T lymphocyte, wherein said lymphocyte comprises:
an antigen-specific receptor, wherein the presence of said antigen-specific receptor leads to increased in vivo survival of said lymphocyte; and
an antitumor chimeric receptor; and
administering said T lymphocyte to said individual.

23. The method of claim 20 or 22, wherein said antigen is an Epstein-Barr Virus polypeptide.

24. The method of claim 18, 20, or 22, wherein said obtaining step is further defined as:
transfecting into a T lymphocyte a vector comprising a polynucleotide encoding said chimeric receptor.

25. The method of claim 24, wherein said vector is a retroviral vector.

26. The method of claim 22, wherein said antitumor chimeric receptor is 14.G2a-ζ.

27. The method of claim 22, wherein said antitumor chimeric receptor is CD-19 specific.

28. The method of claim 22, wherein the tumor is of neural crest origin.

29. The method of claim 28, wherein the tumor of neural crest origin is neuroblastoma or ganglioneuroma.

30. The method of claim 22, wherein the tumor is from lung cancer, melanoma, breast cancer, prostate cancer, colon cancer, or lymphoma.

31. The method of claim 30, wherein the lymphoma is of B cell origin.

32. The method of claim 22, further comprising administering to said individual an additional cancer therapy.

33. The method of claim 32, wherein said additional cancer therapy is chemotherapy, radiation, surgery, or a combination thereof.

34. A method of preventing cancer or an intractable infection in an individual, wherein said cancer or intractable infection is associated with a pathogen or cell having a first antigen, comprising administering to an individual susceptible to said cancer or intractable infection at least one cytotoxic T lymphocyte, wherein the lymphocyte comprises:
a receptor specific for a second antigen, wherein the presence of said second antigen-specific receptor leads to increased in vivo survival of said lymphocyte; and
a chimeric receptor specific for said first antigen.

35. The method of claim 34, wherein said second antigen is an Epstein-Barr Virus polypeptide.

36. The method of claim 34, wherein said cancer is of neural crest origin.

37. The method of claim 34, wherein said cancer is lung cancer, melanoma, breast cancer, prostate cancer, colon cancer, or lymphoma.

38. The method of claim 37, wherein the lymphoma is of B cell origin.

39. The method of claim 34, wherein said intractable infection is a viral infection or a bacterial infection.

40. The method of claim 38, wherein said viral infection is acquired immunodeficiency syndrome (AIDS), hepatitis B or hepatitis C.

41. A kit, housed in a suitable container, comprising:
at least one cytotoxic T lymphocyte in a pharmaceutically acceptable solution, comprising:
a chimeric receptor specific for said first antigen; and
a receptor specific for a second antigen, wherein the presence of said second antigen-specific receptor leads to increased in vivo survival of said lymphocyte.

42. The kit of claim 41, wherein said second antigen is an Epstein-Barr Virus polypeptide.
